(19) United States
(12) Patent Application Publication
(10) Pub. No.: US 2022/0387605 A1
(43) Pub. Date: Dec. 8, 2022

(54) COMPOSITIONS AND METHODS COMPRISING SELF-ASSEMBLING PEPTIDE-POLYMER NANOFIBERS FOR SUBLINGUAL IMMUNIZATION

(71) Applicant: Duke University, Durham, NC (US)

(72) Inventors: Joel H. Collier, Durham, NC (US); Sean H. Kelly, Durham, NC (US)

(21) Appl. No.: 16/632,226

(22) PCT Filed: Jul. 18, 2018
(86) PCT No.: PCT/US2018/042762

§ 371 Date: Jan. 17, 2020

Related U.S. Application Data
(60) Provisional application No. 62/533,910, filed on Jul. 18, 2017.

(51) Int. Cl. A61K 47/60; A61K 47/42; A61P 37/00

(52) U.S. Cl. CPC A61K 47/60; A61K 47/42; A61P 37/00

(57) ABSTRACT

Disclosed herein are peptide-polymer conjugates for the delivery of peptide epitopes sublingually to elicit an immune response.

### CROSS-REFERENCE TO RELATED APPLICATIONS

This application claims priority to U.S. Provisional Patent Application No. 62/533,910, filed Jul. 18, 2017, which is incorporated herein by reference in its entirety.

### STATEMENT REGARDING FEDERALLY SPONSORED RESEARCH

This invention was made with government support under grant DGE-1644868 awarded by the National Science Foundation and grant R01A1118182 awarded by the National Institutes of Health. The government has certain rights in the invention.

### FIELD

This disclosure relates to materials and compositions for eliciting an adequate immune response to a peptide antigen.

### INTRODUCTION

A growing class of designer biomaterials is being investigated as potential next generation vaccines for a wide range of diseases and therapies, such as infectious diseases, cancer, and autoimmunity. These materials may be used to elicit immune responses with more tailored phenotypes than traditional vaccines based on inactivated or attenuated pathogens. As this new generation of biomaterial-based vaccines begins to move towards greater clinical prevalence, the choice of delivery route is a consideration to ensure an adequate immune response is elicited.

### SUMMARY

In an aspect, the disclosure relates to a peptide-polymer conjugate. The peptide-polymer conjugate may include a self-assembling domain comprising a polypeptide and having a C-terminal and N-terminal end; a peptide epitope or protein antigen linked to the N-terminal end or the C-terminal end of the self-assembling domain; and a polyethylene glycol (PEG) domain linked to the other of the N-terminal end and the C-terminal end of the self-assembling domain.

In some embodiments, the self-assembling domain comprises a polypeptide of 5 to 40 amino acids. In some embodiments, the self-assembling domain comprises a polypeptide having an amino acid sequence selected from SEQ ID NOs: 1-2 and 19-79. In some embodiments, the self-assembling domain forms at least one alpha helix. In some embodiments, the self-assembling domain forms at least one beta sheet. In some embodiments, the self-assembling domain comprises a polypeptide having an amino acid sequence of SEQ ID NO: 1 (QQKFQFQFEQQ). In some embodiments, the peptide-polymer conjugate comprises a peptide epitope, the peptide epitope comprising a polypeptide of 3 to 50 amino acids. In some embodiments, the peptide-polymer conjugate comprises a protein antigen, the protein antigen comprising a polypeptide of 10 to 500 amino acids.

In some embodiments, the peptide epitope or protein antigen is linked to the N-terminal end of the self-assembling domain via a peptide linker. In some embodiments, the peptide linker comprises a polypeptide of 3 to 10 amino acids. In some embodiments, the peptide linker comprises a polypeptide having an amino acid sequence selected from SEQ ID NO: 4 (SGSG), SEQ ID NO: 9 (Gn wherein n is an integer from 1 to 10), SEQ ID NO: 10 (SGSGn wherein n is an integer from 1 to 10), SEQ ID NO: 11 (GSGS), SEQ ID NO: 12 (SSSS), SEQ ID NO: 13 (GGGS), SEQ ID NO: 14 (GGC), SEQ ID NO: 15 (GGS), SEQ ID NO: 16 ((GGC)8), SEQ ID NO: 17 (G4S)3, and SEQ ID NO: 18 (GGAAY).

In some embodiments, the PEG domain has an average molecular weight of 300-5000 Da. In some embodiments, the PEG domain has an average molecular weight of 3000 Da. In some embodiments, the PEG domain is selected from —(CH2—CH2—O)n—CH2—CH2—OH, —(CH2—CH2—O)n—CH2—CH2—O—C1-4 alkyl, —(O—CH2—CH2)n—OH, and —(O—CH2—CH2)n—O—C1-4 alkyl, wherein n is an integer between 1 and 200. In some embodiments, the PEG domain is linked to the self-assembling domain via a linker.

In some embodiments, the conjugate self-assembles into nanofibers. In some embodiments, the conjugate self-assembles into nanofibers having a length of 50 nm to 50,000 nm. In some embodiments, the nanofibers have uniform width. In some embodiments, the nanofibers have a width of 5-100 nm.

In some embodiments, the conjugate elicits an antibody response upon or after administration to a subject. In some embodiments, the conjugate elicits a T cell response upon or after administration to a subject. In some embodiments, the conjugate is administered to the subject sublingually, orally, intranasally, intravenously, parenterally, subcutaneously, intramuscularly, intraperitoneally, rectally, intravaginally, or intrathecally. In some embodiments, the conjugate is administered to the subject sublingually.

In a further aspect, the disclosure relates to a supramolecular complex comprising a plurality of the peptide-polymer conjugates as detailed herein. In some embodiments, the plurality of peptide-polymer conjugates comprises a plurality of identical peptide-polymer conjugates. In some embodiments, the plurality of peptide-polymer conjugates comprises a plurality of non-identical peptide-polymer conjugates. In some embodiments, the supramolecular complex comprises n different peptide-polymer conjugates, wherein n is an integer from 1 to 10,000. In some embodiments, then different peptide-polymer conjugates comprise different self-assembling domains from each other, different PEG domains from each other, or different peptide epitopes or protein antigens from each other, or a combination thereof. In some embodiments, the supramolecular complex further includes at least one self-assembling domain not linked to a peptide epitope or a protein antigen, or at least one self-assembling domain not linked to a PEG domain, or at least one self-assembling domain not linked to a PEG domain and not linked to a peptide epitope or a protein antigen, or a combination thereof.

Another aspect of the disclosure provides a composition comprising a plurality of the conjugates as detailed herein, and a pharmaceutically acceptable carrier.

Another aspect of the disclosure provides methods of immunizing a subject. The methods may include comprising administering to the subject an effective amount of the conjugate, the supramolecular complex, or the composition as detailed herein.

Another aspect of the disclosure provides methods of eliciting an immune response to a peptide epitope in a subject. The methods may include administering to the subject an effective amount of the conjugate, the supramolecular complex , or the composition as detailed herein.

In some embodiments, the conjugate is administered to the subject sublingually, orally, intranasally, intravenously, parenterally, subcutaneously, intramuscularly, intraperitoneally, rectally, intravaginally, or intrathecally. In some embodiments, the conjugate is administered sublingually. In some embodiments, the conjugate is co-administered with an adjuvant. In some embodiments, the adjuvant comprises cholera toxin, CpG, cyclic-di-GMP, cyclic-di-AMP, or a combination thereof.

The disclosure provides for other aspects and embodiments that will be apparent in light of the following detailed description and accompanying figures.

### BRIEF DESCRIPTION OF THE DRAWINGS

FIG. 1A, FIG. 1B, FIG. 1C. OVAQ11-PEG self-assembled into β-sheet nanofibers. Negative stained TEM images of self-assembled (FIG. 1A) OVAQ11 and (FIG. 1B) OVAQ11-PEG nanofibers assembled from 2 mM peptide (19000× magnification; scale bar is 500 nm). (FIG. 1C) Circular dichroism of peptides assembled at 3 mM in PBS and diluted to 0.1 mM in potassium fluoride just prior to analyzing.

FIG. 2A, FIG. 2B, FIG. 2C, FIG. 2D. Fluorescent probes show a critical aggregation concentration near 1 mM for self-assembly of OVAQ11-PEG into nanofibers. OVAQ11-PEG was dissolved in PBS containing either thioflavin T, which binds to amyloid structures, or pyrene, which is sensitive to changes in hydrophobicity during self-assembly, at various concentrations. (FIG. 2A) and (FIG. 2C) show the linear relationship between peptide concentration and fluorescence of thioflavin T and pyrene, respectively. (FIG. 2B) and (FIG. 2D) present the same data with a log scale x-axis to determine the critical aggregation concentration, which is the concentration corresponding to the intersection of the pre- and post-assembly tangent lines (circled).

FIG. 3A, FIG. 3B, FIG. 3C, FIG. 3D. Shearing of OVAQ11 produces nanofibers with a mean length below the average pore size of mucus. Ten TEM images of OVAQ11 were obtained before and after shearing through a 100 nm track-etched polycarbonate membrane using an Avanti mini-extruder. ImageJ was used to determine the length of 450 individual sheared fibers. Representative images of unsheared (FIG. 3A) and sheared (FIG. 3B) OVAQ11 fibers (scale bar is 500 nm). (FIG. 3C) ImageJ fiber trace corresponding to the image in FIG. 3B. (FIG. 3D) Histogram showing the frequency distribution of fiber lengths after shearing.

FIG. 4A, FIG. 4B, FIG. 4C. PEGylation and shearing do not impact the immunogenicity of OVAQ11. (FIG. 4A) Presentation of the pOVA epitope in MHC class II molecules to T cells was measured in vitro by culturing nanofibers with mouse bone-marrow derived dendritic cells for 2 hours, followed by washing and overnight culture with the DOBW reporter T cell line. IL-2 is released by the DOBW cells upon encountering DC with pOVA-loaded MHC II, and IL-2 concentration in the supernatant was measured by ELISA. (FIG. 4B) Mice were immunized subcutaneously on weeks 0 and 4 with two 50 μL injections of 2 mM peptide, and serum was analyzed by ELISA for antibodies against the immunizing peptide. (FIG. 4C) Distribution of pOVA-specific antibodies isotypes in week 7 serum of mice from FIG. 4B. Data were analyzed by one-way ANOVA with repeated measures.

FIG. 5A, FIG. 5B. OVAQ11-PEG raises antigen-specific humoral responses after sublingual immunization. (FIG. 5A) Adjuvant plus peptide alone fails to elicit sublingual antibody responses. Mice were immunized at days 0, 7, and 21 with 7 μL of 5.6 mM peptide with or without 2 μg of cholera toxin (CT) adjuvant, and pOVA-specific IgG was measures by ELISA at week 6. (FIG. 5B) Mice were immunized according to the same schedule as in FIG. 5A. Nanofibers in sheared groups were shortened by passage through a 100 nm track-etched polycarbonate membrane using an Avanti mini extruder. Data in FIG. 5B were analyzed by one-way ANOVA with repeated measures.

FIG. 6A, FIG. 6B, FIG. 6C, FIG. 6D. PEGylation of OVAQ11 nanofibers, but not shearing, is a factor for inducing antibody responses after sublingual immunization. (FIG. 6A) Mice were immunized at days 0, 7, 21, and 140 with 7 μL of 5.6 mM peptide with no adjuvant or with 2 μg of cholera toxin, and pOVA-specific IgG was measured by ELISA. Nanofibers in sheared groups were shortened by passage through a 100 nm track-etched polycarbonate membrane using an Avanti mini extruder. (FIG. 6B) Titer data from sheared and unsheared cholera toxin groups from FIG. 6A were combined and re-presented to highlight the effect of PEGylation. (FIG. 6C) Week 21 titer data from OVAQ11-PEG plus cholera toxin groups in FIG. 6A and OVAQ11 plus cholera toxin from FIG. 5B are re-presented to highlight the effects of shearing. Data in FIG. 6A and FIG. 6B were analyzed by one-way ANOVA with repeated measures. Data in FIG. 6C were analyzed by multiple t-tests with Bonferroni correction. (FIG. 6D) Sheared nanofibers were prepared by passage through a 100 nm track-etched polycarbonate membrane using an Avanti mini extruder. This method significantly reduced the length of the nanofibers from hundreds of nanometers long to around 100 nm. This shearing did not improve the sublingual immunogenicity of non-PEGylated OVAQ11 nanofibers (left), nor did it significantly diminish the sublingual immunogenicity of PEGylated OVAQ11 nanofibers (right).

FIG. 7. OVAQ11-PEG elicits antigen-specific T-cell responses after sublingual immunization. Mice from FIG. 5B and FIG. 6A were sacrificed at week 21 (one week after the final boost). Spleens were harvested and pOVA-specific T cells responses were measured by ELISPOT. The number of IFNγ secreting cells (spot-forming cells, SFC) were quantified.

FIG. 8A, FIG. 8B. Mucosal antibody responses after sublingual immunization. Mice were immunized at weeks 0, 1, 3, and 9 with 7 μL of 5.6 mM peptide in 1X PBS with 2 μg cholera toxin adjuvant. Mice were sacrificed at week 10 (one week after the final immunization), and vaginal (FIG. 8A) and nasal (FIG. 8B) washes were collected. Samples were diluted 1:10 and pOVA-specific IgG was measured by ELISA. Data was analyzed by student's t-test.

FIG. 9. Strong antibody response are elicited with CTB adjuvant. Mice were immunized at weeks 0, 1, 3, and 6, with 7 μL of 5.6 mM peptide with 10 μg of cholera toxin subunit B, and pOVA-specific IgG was measured by ELISA.

FIG. 10. Artistic rendering of proposed structure of a peptide-polymer conjugate (top) and peptide-polymer conjugates formed into a supramolecular complex or nanofiber (bottom).

FIG. 11A, FIG. 11B. Properties of OVAQ11-PEG. (FIG. 11A) MALDI spectrum showing the molecular weight distribution of OVAQ11-PEG, due to the polydisperse PEG block on the C-terminal. (FIG. 11B) Zeta-potentials of OVAQ11-PEG and OVAQ11. Peptides were prepared at 2 mM in 1X PBS and diluted to 0.2 mM in 1X PBS prior to measurement at 25° C.

FIG. 12A, FIG. 12B. Quantification of length distribution of sheared OVAQ11. (FIG. 11A) Negative stained TEM images of sheared OVAQ11 and accompanying nanofiber traces created in ImageJ. (FIG. 11B) Representative negative stained TEM images of unsheared OVAQ11.

FIG. 13. Effect of shearing on the concentration of Q11 nanofiber solutions. Tryptophan labelled Q11 (W-Q11) was prepared at 2 mM in 1X PBS and sheared through a 100 nm laser track-etched polycarbonate membrane using an Avanti Polar Lipids mini extruder. The absorbance at 210, 215, and 280 nm were recorded before and after shearing. Replicates are from three independently sheared solutions.

FIG. 14. Antibody response after sublingual immunization with adjuvanted ovalbumin protein. Mice (n=5) were immunized sublingually on days 0, 7, and 14 with 200 μg of ovalbumin protein and 2 μg of cholera toxin in a total volume of 5 μL of 1X PBS. Ovalbumin specific IgG in sera was measured by ELISA.

FIG. 15. Complete ELISPOT results. Results from two independent ELISPOT assays are shown. Cells were stimulated with pOVA, left unstimulated as a negative control (Cells Only), or stimulated with Concanavalin A as a positive control (ConA), and the number of IFNγ secreting cells (spot-forming cells, SFC) were quantified. For clarity, FIG. 7 reports only the results of cells stimulated with pOVA. SFC: spot-forming cells.

### DETAILED DESCRIPTION

Described herein are peptide-polymer conjugates that form nanofibers and may be used to elicit an immune response in a subject. The peptide-polymer conjugate includes a peptide epitope or protein antigen, to which the immune response is elicited. The peptide-polymer conjugate includes a self-assembling domain comprising a polypeptide and having a C-terminal and N-terminal end, the peptide epitope or protein antigen linked to the N-terminal end or the C-terminal end of the self-assembling domain, and a polyethylene glycol (PEG) domain linked to the other of the N-terminal end and the C-terminal end of the self-assembling domain. A plurality of the peptide-polymer conjugates may form a supramolecular complex. The peptide-polymer conjugate, or a supramolecular complex thereof, may facilitate sublingual administration of the peptide epitope or protein antigen to a subject. The peptide-polymer conjugate, or a supramolecular complex thereof, may be used to immunize a subject or elicit an immune response in a subject. The peptide-polymer conjugate, or a supramolecular complex thereof, may provide additional advantages over traditional vaccines such as for example, increased specificity of targeted epitopes.

Sublingual immunization offers advantages over other delivery routes such as intramuscular, intraperitoneal, or subcutaneous, in that it is less invasive and likely to result in better patient compliance. Peptide epitopes offer advantages over whole protein immunogens because the desired epitope can be defined precisely. However, peptides are not strongly immunogenic sublingually. For example the model peptide epitope ovalbumin₃₂₃₋₃₃₉ (pOVA) fails to elicit responses after sublingual immunization, even when formulated with the adjuvant cholera toxin. The present disclosure addresses these problems by providing supramolecular peptide-polymer conjugates that can elicit immune responses after sublingual immunization. The PEG-peptide conjugates detailed herein self-assemble into long nanofibers with, for example, uniform widths, which can elicit antibody responses when delivered sublingually. The delivery of a biomaterials vaccine via the sublingual route presents potential immunological, logistical, and financial advantages over conventional needle-based immunizations.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “alkyl” as used herein, means a straight or branched, saturated hydrocarbon chain containing from 1 to 20 carbon atoms. The term “C₁₋₁₂ alkyl” means a straight or branched chain hydrocarbon containing from 1 to 12 carbon atoms. The term “lower alkyl” or “C₁₋₄ alkyl” means a straight or branched chain hydrocarbon containing from 1 to 4 carbon atoms. The term “C₁₋₃ alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl” as used herein, means an unsaturated hydrocarbon chain containing from 2 to 20 carbon atoms and at least one carbon-carbon double bond.

The term “amino” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl. In some embodiments, amino is —NH₂.

The term “carboxyl” as used herein, means a carboxylic acid, or—COON.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “adjuvant” refers to a compound, conjugate, or composition that enhances the immune response to an antigen. An adjuvant is distinct from the target antigen and is capable of enhancing or potentiating immune effector cell activation. Adjuvants may contain a substance to protect the antigen from rapid catabolism, such as aluminum hydroxide or a mineral oil, and also a protein derived from lipid A, Bortadella pertussis, or Mycobacterium tuberculosis. Suitable adjuvants may be commercially available and include, for example, complete or incomplete Freund's adjuvant; AS-2; aluminum salts such as aluminum hydroxide (as a gel, where appropriate) or aluminum phosphate; calcium salts, iron salts, or zinc salts; an insoluble suspension of acylated tyrosine; acylated sugars; cationically or anionically derivatized polysaccharides; polyphosphazenes; biologically degradable microspheres; monophosphoryl lipid A, cytokines such as GM-CSF, Interleukin-2, Interleukin-7, and Interleukin-12; cholera toxin, CpG, cyclic-di-GMP, and cyclic-di-AMP.

The term “administration” or “administering,” as used herein, refers to providing, contacting, and/or delivery of an agent by any appropriate route to achieve the desired effect. These agents may be administered to a subject in numerous ways including, but not limited to, sublingually, orally, ocularly, nasally, intravenously, topically, as aerosols, suppository, etc. and may be used in combination.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, N.C.). The healthy or normal levels or ranges for a target or for a protein activity may be defined in accordance with standard practice. A control may be a subject, or a sample therefrom, whose disease state is known. The subject, or sample therefrom, may be healthy, diseased, diseased prior to treatment, diseased during treatment, or diseased after treatment, or a combination thereof. The term “normal subject” as used herein means a healthy subject, i.e. a subject having no clinical signs or symptoms of disease. The normal subject may be clinically evaluated for otherwise undetected signs or symptoms of disease, which evaluation may include routine physical examination and/or laboratory testing. In some embodiments, the control is a healthy control.

“Antigen” refers to a molecule capable of being bound by an antibody or a T cell receptor. The term “antigen”, as used herein, also encompasses T-cell epitopes. An antigen is additionally capable of being recognized by the immune system and/or being capable of inducing a humoral immune response and/or cellular immune response leading to the activation of B-lymphocytes and/or T-lymphocytes. In some embodiments, the antigen contains or is linked to a Th cell epitope. An antigen can have one or more epitopes (B- epitopes and T-epitopes). Antigens may include peptides, polypeptides, polynucleotides, carbohydrates, lipids, small molecules, and combinations thereof. Antigens may also be mixtures of several individual antigens. “Antigenicity” refers to the ability of an antigen to specifically bind to a T cell receptor or antibody and includes the reactivity of an antigen toward pre-existing antibodies in a subject. “Immunogenicity” refers to the ability of any antigen to induce an immune response and includes the intrinsic ability of an antigen to generate antibodies in a subject.

“Polymer” or “synthetic polymer” refers to a polymer which is produced from at least one monomer by a chemical process. A synthetic polymer is not produced directly by a living organism. Synthetic polymers include a homopolymer, heteropolymer, block polymer, co-polymer, ter-polymer, etc., and blends, combinations and mixtures thereof. Examples of synthetic polymers include, but are not limited to, functionalized polymers, such as a polymer comprising 5-vinyltetrazole monomer units and having a molecular weight distribution less than 2.0. A synthetic polymer may be or contain one or more of a star block copolymer, a linear polymer, a branched polymer, a hyperbranched polymer, a dendritic polymer, a comb polymer, a graft polymer, a brush polymer, a bottle-brush copolymer and a crosslinked structure, such as a block copolymer comprising a block of 5-vinyltetrazole monomer units. Synthetic polymers include, without limitation, polyesters, poly(meth)acrylamides, poly(meth)acrylates, polyethers, polystyrenes, polynorbornenes and monomers that have unsaturated bonds. For example, amphiphilic comb polymers are described in U.S. Patent Application Publication No. 2007/0087114 and in U.S. Pat. No. 6,207,749 to Mayes et al., the disclosure of each of which is herein incorporated by reference in its entirety. The amphiphilic comb-type polymers may be present in the form of copolymers, containing a backbone formed of a hydrophobic, water-insoluble polymer and side chains formed of short, hydrophilic non-cell binding polymers. Examples of other synthetic polymers include, but are not limited to, polyalkylenes such as polyethylene and polypropylene and polyethyleneglycol (PEG); polychloroprene; polyvinyl ethers; such as poly(vinyl acetate); polyvinyl halides such as poly(vinyl chloride); polysiloxanes; polystyrenes; polyurethanes; polyacrylates; such as poly(methyl (meth)acrylate), poly(ethyl (meth)acrylate), poly(n-butyl(meth)acrylate), poly(isobutyl (meth)acrylate), poly(tert-butyl (meth)acrylate), poly(hexyl(meth)acrylate), poly(isodecyl (meth)acrylate), poly(lauryl (meth)acrylate), poly(phenyl (meth)acrylate), poly(methyl acrylate), poly(isopropyl acrylate), poly(isobutyl acrylate), and poly(octadecyl acrylate); polyacrylamides such as poly(acrylamide), poly(methacrylamide), poly(ethyl acrylamide), poly(ethyl methacrylamide), poly(N-isopropyl acrylamide), poly(n, iso, and tert-butyl acrylamide); and copolymers and mixtures thereof. These synthetic polymers may include useful derivatives, including synthetic polymers having substitutions, additions of chemical groups, for example, alkyl groups, alkylene groups, hydroxylations, oxidations, and other modifications routinely made by those skilled in the art. The synthetic polymers may include zwitterionic polymers such as, for example, polyphosphorycholine, polycarboxybetaine, and polysulfobetaine. The synthetic polymers may have side chains of betaine, carboxybetaine, sulfobetaine, oligoethylene glycol (OEG), sarcosine or polyethyleneglycol (PEG). Synthetic polymers may include polyethyleneglycol (PEG).

“Polynucleotide” as used herein can be single stranded or double stranded, or can contain portions of both double stranded and single stranded sequences. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be, for example, 2 to 20, 2 to 15, or 2 to 10 amino acids in length. In some embodiments, a motif includes 3, 4, 5, 6, or 7 sequential amino acids. A domain may be comprised of a series of the same type of motif.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. In some embodiments, a carrier includes a solution at neutral pH. In some embodiments, a carrier includes a salt. In some embodiments, a carrier includes a buffered solution. In some embodiments, a carrier includes phosphate buffered saline solution.

“Recombinant” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all. The term “host cell” is a cell that is susceptible to transformation, transfection, transduction, conjugation, and the like with a nucleic acid construct or expression vector. Host cells can be derived from plants, bacteria, yeast, fungi, insects, animals, etc. In some embodiments, the host cell includes Escherichia coli.

“Sample” or “test sample” as used herein can mean any sample in which the presence and/or level of a target, agent, or activity is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. Samples may be obtained before treatment, before diagnosis, during treatment, after treatment, or after diagnosis, or a combination thereof.

“Subject” as used herein can mean a mammal that wants or is in need of the herein described therapies and compositions. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant. In some embodiments, the subject is human. In some embodiments, the subject has a specific genetic marker.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides.

A “therapeutically effective amount,” or “effective dosage,” or “effective amount” as used interchangeably herein unless otherwise defined, means a dosage of an agent or drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in a subject. A therapeutically effective amount may be administered in one or more administrations (e.g., the composition may be given as a preventative treatment or therapeutically at any stage of disease progression, before or after symptoms, and the like), applications, or dosages, and is not intended to be limited to a particular formulation, combination, or administration route. It is within the scope of the present disclosure that the drug may be administered at various times during the course of treatment of the subject. The times of administration and dosages used will depend on several factors, such as the goal of treatment, condition of the subject, etc. and can be readily determined by one skilled in the art. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, the term “toxic” refers to an amount of a chemical entity, agent, or substance that would be harmful to the subject or cause any adverse effect. The term “non-toxic” refers to a substance that has a relatively low degree to which it can damage a subject. “Cytotoxic” refers to a chemical entity, agent, or substance that is toxic to cells. Toxicity can refer to the effect on a whole organism, such as an animal, bacterium, plant, or other subject as defined herein, as well as the effect on a substructure of the organism, such as a cell (cytotoxicity) or an organ (organotoxicity), such as the liver (hepatotoxicity). A central concept of toxicology is that effects are dose-dependent; even water can lead to water intoxication when taken in large enough doses, whereas for even a very toxic substance such as snake venom there is a dose below which there is no detectable toxic effect. A composition or compound that is relatively non-toxic may allow a wider range of subjects to be able to safely handle the composition or compound, without serious safety concerns or risks.

The terms “treat,” “treated,” or “treating” as used herein refers to a therapeutic wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment. The terms “treat,” “treated,” or “treating” may include suppressing, repressing, ameliorating, or completely eliminating the disease. Preventing the disease may involve administering a conjugate, complex, or composition of the present invention to a subject prior to onset of the disease. Suppressing the disease may involve administering a conjugate, complex, or composition of the present invention to a subject after induction of the disease but before its clinical appearance. Repressing or ameliorating the disease may involve administering a conjugate, complex, or composition of the present invention to a subject after clinical appearance of the disease.

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide, to bind a ligand, or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence ora fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence ora fragment thereof. In some embodiments, variants include homologues. Homologues may be polynucleotides or polypeptides or genes inherited in two species by a common ancestor.

### 2. Peptide-Polymer Conjugate

Provided herein is a peptide-polymer conjugate. The peptide-polymer conjugate includes a self-assembling domain, a peptide epitope or protein antigen, and a polyethylene glycol (PEG) domain. Peptide-polymer conjugates may co-assemble into nanofibers or supramolecular complexes.

### a. Self-assembling Domain

The peptide-polymer conjugate includes a self-assembling domain. As used herein, the term “self-assembling domain” refers to peptides that are able to spontaneously associate and form stable structures. The self-assembling domain may also be referred to as a self-assembling peptide. The self-assembling domain may include a polypeptide and have a C-terminal and N-terminal end. In some embodiments, the self-assembling domain has a neutral net charge. In some embodiments, the self-assembling domain comprises a polypeptide having alternating hydrophobic and hydrophilic amino acids. Hydrophobic amino acids include, for example, Ala, Val, Ile, Leu, Met, Phe, Tyr, Trp, Cyc, and Pro. Hydrophillic amino acids include, for example, Arg, His, Lys, Asp, Glu, Ser, Thr, Asn, and Gln. In some embodiments, the self-assembling domain is glutamine-rich. A glutamine-rich self-assembling domain may comprise a polypeptide wherein at least about 50%, at least about 55%, at least about 60%, at least about 65%, at least about 70%, at least about 75%, at least about 80%, at least about 85%, or at least about 90% of the amino acids are glutamine. The self-assembling domain comprises a polypeptide of 5 to 40 amino acids. The self-assembling peptide may include at least, at most, or exactly 5, 10, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids.

In some embodiments, the self-assembling domain may form or comprises at least one alpha helix. In some embodiments, the self-assembling domain may form or comprises at least one beta sheet. Examples of self-assembling domains are shown in TABLE 1. Self-assembling domains or peptides are also detailed in PCT/US2017/025596 (filed 31 Mar. 2017 and published 5 Oct. 2017 as WO 2017/0173398) and Bowerman et al. (Biopolymers (Peptide Science) 2012, 98, 169-184), which are incorporated herein by reference. In some embodiments, the self-assembling domain comprises a polypeptide selected from the sequences listed in TABLE 1. In some embodiments, the self-assembling domain comprises a polypeptide having an amino acid sequence of SEQ ID NO: 1 (QQKFQFQFEQQ), or a polypeptide with at least 75%, at least 80%, at least 85%, at least 90%, or at least 95% identity thereto.

TABLE 1
Examples of self-assembling domains.

| Name | Peptide Sequence | Structure | SEQ ID NO. |
|---|---|---|---|
| Q11 | QQKFQFQFEQQ | beta-sheet | 1 |
| W-Q11 | WQQKFQFQFEQQ | beta-sheet | 2 |
| | ZₙbXXXbZₘ wherein b is independently any | alpha-helix | 19 |
| | positively charged amino acid, Z is | | |
| | independently any amino acid, X is | | |
| | independently any amino acid, n is an | | |
| | integer from 0 to 20, and m is an integer | | |
| | from 0 to 20. | | |
| | QARILEADAEILRAYARILEAHAEILRAQ | alpha-helix | 20 |
| | QAKILEADAEILKAYAKILEAHAEILKAQ | alpha-helix | 21 |
| | ADAEILRAYARILEAHAEILRAQ | alpha-helix | 22 |
| EAK16-I | Ac-(AEAKAEAK)₂-NH₂ | beta-sheet | 23 |
| EAK16-II | Ac-(AEAEAKAK)₂-NH₂ | beta-sheet | 24 |
| EAK16-IV | Ac-(AEAE)₂(AKKE)₂-NH₂ | beta-sheet | 25 |
| EMK16-II | Ac-(MEMEMKMK)-NH₂ | beta-sheet | 26 |
| RAD16-I | Ac-(RADARADA)₂-NH₂ | beta-sheet | 27 |
| RAD16-II | Ac-(RARARDRD)₂-NH₂ | beta-sheet | 28 |
| RAD16-IV | Ac-(RARA)₂(RDRD)₂-NH₂ | beta-sheet | 29 |
| DAR16-IV | Ac-(ADAD)₂(ARAR)₂-NH₂ | beta-sheet | 30 |
| RAD8-I | Ac-(RADARADA)-NH₂ | random coil | 31 |
| RF6 | Ac-(RFRFRF)-NH₂ | random coil | 32 |
| RF8 | Ac-(RFRFRFRF)-NH₂ | random coil | 33 |
| KLD16 | Ac-(KLDL)₄-NH₂ | beta-sheet | 34 |
| FKFE2 | Ac-(FKFE)₂-NH₂ | beta-sheet | 35 |
| EFK12 | Ac-(FKFE)₃-NH₂ | beta-sheet | 36 |
| EFK16 | Ac-(FEFEFKFK)₂-NH₂ | beta-sheet | 37 |
| MAX1 | H₂N-VKVKVKVK-V^{D}PPT-KVKVKVKV-NH₂ | | 38 |
| MAX2 (V16T) | H₂N-VKVKVKVK-V^{D}PPT-KVKTKVKV-NH₂ | | 39 |
| MAX3 (V7T, | H₂N-VKVKVKTK-V^{D}PPT-KVKTKVKV-NH₂ | | 40 |
| V16T) | | | |
| MAX4 | H₂N-KVKVKVKV-K^{D}PPS-VKVKVKVK-NH₂ | | 41 |
| MAX5 (T12S) | H₂N-VKVKVKVK-V^{D}PPS-KVKVKVKV-NH₂ | | 42 |
| MAX6 (V16E) | H₂N-VKVKVKVK-V^{D}PPT-KVKEKVKV-NH₂ | | 43 |
| MAX7 (V16C) | H₂N-VKVKVKVK-V^{D}PPT-KVKCKVKV-NH₂ | | 44 |
| MAX8 (K15E) | H₂N-VKVKVKVK-V^{D}PPT-KVEVKVKV-NH₂ | | 45 |
| (K2E) | H₂N-VEVKVKVK-V^{D}PPT-KVKVKVKV-NH₂ | | 46 |
| (K4E) | H₂N-VKVEVKVK-V^{D}PPT-KVKVKVKV-NH₂ | | 47 |
| (K6E) | H₂N-VKVKVEVK-V^{D}PPT-KVKVKVKV-NH₂ | | 48 |
| (K8E) | H₂N-VKVKVKVE-V^{D}PPT-KVKVKVKV-NH₂ | | 49 |
| (K13E) | H₂N-VKVKVKVK-V^{D}PPT-EVKVKVKV-NH₂ | | 50 |
| (K17E) | H₂N-VKVKVKVK-V^{D}PPT-KVKVEVKV-NH₂ | | 51 |
| P₁₁-1 | Ac-QQRQQQQQEQQ-NH₂ | beta-sheet | 52 |
| P₁₁-2 | Ac-QQRFQWQFEQQ-NH₂ | beta-sheet | 53 |
| P₁₁-3 | Ac-QQRFQWQFQQQ-NH₂ | beta-sheet | 54 |
| P₁₁-4 | Ac-QQRFEWEFEQQ-NH₂ | beta-sheet | 55 |
| P₁₁-5 | Ac-QQOFOWOFQQQ-NH₂ | beta-sheet | 56 |
| P₁₁-7 | Ac-SSRFSWSFESS-NH₂ | beta-sheet | 57 |
| P₁₁-8 | Ac-QQRFOWOFEQQ-NH₂ | beta-sheet | 58 |
| P₁₁-9 | Ac-SSRFEWEFESS-NH₂ | beta-sheet | 59 |
| P₁₁-12 | Ac-SSRFOWOFESS-NH₂ | beta-sheet | 60 |
| P₁₁-14 | Ac-QQOFOWOFOQQ-NH₂ | unstructured | 61 |
| P₁₁-I6 | Ac-NNRFOWOFEQQ-NH₂ | beta-sheet | 62 |
| P₁₁-18 | Ac-TTRFOWOFETT-NH₂ | beta-sheet | 63 |
| P₁₁-19 | Ac-QQRQOQOQEQQ-NH₂ | beta-sheet | 64 |
| 1 | Ac-FEFEFKFKFEFEFKFK-NH₂ | | 65 |
| 2 | Ac-FEFEAKFKFEFEFKFK-NH₂ | | 66 |
| 3 | Ac-FEFEFKLKIEFEFKFK-NH₂ | | 67 |
| 4 | Ac-FEAEVKLKIELEVKFK-NH₂ | | 68 |
| 5 | Ac-GEAEVKLKIELEVKAK-NH₂ | | 69 |
| 6 | Ac-GEAEVKIKIEVEAKGK-NH₂ | | 70 |
| 7 | Ac-IEVEAKGKGEAEVKIK-NH₂ | | 71 |
| 8 | Ac-IELEVKAKGEAEVKLK-NH₂ | | 72 |
| 9 | Ac-IELEVKAKAEAEVKLK-NH₂ | | 73 |
| 10 | Ac-IEAEGKGKIEGEAKIK-NH₂ | | 74 |
| 11 | Ac-K₂(QL)₆K₂-NH₂ | | 75 |
| 12 | Ac-E(QL)₆E-NH₂ | | 76 |
| 13 | Ac-K₂(SL)₆K₂-NH₂ | | 77 |
| 14 | Ac-E(SL)₆E-NH₂ | | 78 |
| 15 | Ac-E(CLSL)₃E-NH₂ | | 79 |

In some embodiments, the self-assembling domain includes a modification to the C-terminus, to the N-terminus, or to both the C-terminus and N-terminus. N-terminal modifications may include, for example biotin and acetyl. C-terminal modifications may include, for example, amino and amide. In some embodiments, modifications to the C-terminus and/or to the N-terminus include those shown in TABLE 1 and as described in Bowerman et al. (Biopolymers (Peptide Science) 2012, 98, 169-184). In some embodiments, the self-assembling domain comprises a polypeptide selected from those listed in TABLE 1 but excluding a N-terminal and/or C-terminal modification shown in the table.

### b. Peptide Epitope or Protein Antigen

The peptide-polymer conjugate includes a peptide epitope or protein antigen. In some embodiments, the peptide-polymer conjugate comprises a peptide epitope. The peptide epitope may comprise a polypeptide of 3 to 50 amino acids. The peptide epitope may be linked to the N-terminal end or the C-terminal end of the self-assembling domain. In some embodiments, the peptide epitope is linked to the N-terminal end of the self-assembling domain. In some embodiments, the peptide epitope is linked to the C-terminal end of the self-assembling domain. In some embodiments, the peptide epitope is immunogenic. In some embodiments, the peptide epitope is antigenic.

In some embodiments, the peptide-polymer conjugate comprises a protein antigen. The protein antigen may comprise a polypeptide of 10 to 500 amino acids. In some embodiments, the peptide epitope is comprised within a protein antigen. In some embodiments, the peptide epitope is a portion of a protein antigen. The protein antigen may be linked to the N-terminal end or the C-terminal end of the self-assembling domain. In some embodiments, the protein antigen is linked to the N-terminal end of the self-assembling domain. In some embodiments, the protein antigen is linked to the C-terminal end of the self-assembling domain. In some embodiments, the protein antigen is immunogenic. In some embodiments, the protein antigen is antigenic.

The peptide epitope or protein antigen can be any type of biologic molecule or a portion thereof. Common categories of antigens include, but are not limited to, viral antigens, bacterial antigens, fungal antigens, protozoa and other parasitic antigens, tumor antigens, antigens involved in autoimmune disease, allergy and graft rejection, and other miscellaneous antigens. Antigens can be microbial antigens, such as viral, fungal, or bacterial; or therapeutic antigens such as antigens associated with cancerous cells or growths, or autoimmune disorders. In some embodiments, the peptide epitope comprises a B cell epitope or T cell epitope. In some embodiments, the peptide epitope comprises a B cell epitope and a T cell epitope. In some embodiments, the peptide epitope or protein antigen comprises an autologous target or a portion thereof. In some embodiments, the peptide epitope or protein antigen comprises a cytokine or a portion thereof.

Viral Antigens. Examples of viral antigens include, but are not limited to, retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B. and C, viral components such as hepatitis C viral RNA; influenza viral antigens such as hemagglutinin and neuraminidase and other influenza viral components; measles viral antigens such as the measles virus fusion protein and other measles virus components; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion protein, the M2 protein and other respiratory syncytial viral antigen components; herpes simplex viral antigens such as immediate early proteins, glycoprotein D, and other herpes simplex viral antigen components; varicella zoster viral antigens such as gpl, gpll, and other varicella zoster viral antigen components; Japanese encephalitis viral antigens such as proteins E, M-E, M-E-NS 1, NS 1, NS 1-NS2A, 80% E, and other Japanese encephalitis viral antigen components; rabies viral antigens such as rabies glycoprotein, rabies nucleoprotein and other rabies viral antigen components; or a portion thereof. See Fundamental Virology, Second Edition, e′s. Fields, B. N. and Knipe, D. M. (Raven Press, New York, 1991) for additional examples of viral antigens.

Bacterial Antigens. Bacterial antigens may include, but are not limited to, pertussis bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; hemophilus influenza bacterial antigens such as capsular polysaccharides and other hemophilus influenza bacterial antigen components; anthrax bacterial antigens such as anthrax protective antigen and other anthrax bacterial antigen components; rickettsiae bacterial antigens such as romps and other rickettsiae bacterial antigen component, or a portion thereof. Also included with the bacterial antigens described herein are any other bacterial, mycobacterial, mycoplasmal, rickettsial, or chlamydial antigens; or a portion thereof.

Fungal Antigens. Fungal antigens may include, but are not limited to, Candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components; or a portion thereof.

Parasite Antigens. Examples of protozoa and other parasitic antigens may include, but are not limited to, plasmodium falciparum antigens such as merozoite surface antigens, sporozoite surface antigens, circumsporozoite antigens, gametocyte/gamete surface antigens, blood-stage antigen pf 1 55/RESA and other plasmodial antigen components; toxoplasma antigens such as SAG-1, p30 and other toxoplasma antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components; or a portion thereof.

Tumor antigens. Tumor antigens may include, but are not limited to, telomerase components; multidrug resistance proteins such as P-glycoprotein; MAGE-1, alpha fetoprotein, carcinoembryonic antigen, mutant p53, immunoglobulins of B-cell derived malignancies, fusion polypeptides expressed from genes that have been juxtaposed by chromosomal translocations, human chorionic gonadotrpin, calcitonin, tyrosinase, papillomavirus antigens, gangliosides or other carbohydrate-containing components of melanoma or other tumor cells; or a portion thereof. It is contemplated that antigens from any type of tumor cell can be used in the compositions and methods described herein.

Antigens Relating to Autoimmunity. Antigens involved in autoimmune diseases, allergy, and graft rejection can be used in the compositions and methods. For example, an antigen involved in any one or more of the following autoimmune diseases or disorders can be used: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy may include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection may include antigenic components of the graft to be transplanted into the graft recipient such as heart, lung, liver, pancreas, kidney, and neural graft components. An antigen can also be an altered peptide ligand useful in treating an autoimmune disease.

Examples of miscellaneous antigens which can be can be used in the compositions and methods include endogenous hormones such as luteinizing hormone, follicular stimulating hormone, testosterone, growth hormone, prolactin, and other hormones, drugs of addiction such as cocaine and heroin, and idiotypic fragments of antigen receptors such as Fab-containing portions of an anti-leptin receptor antibody; or a portion thereof.

The peptide epitope or protein antigen may be conjugated or coupled to a self-assembling domain by any means known in the art, including, for example, click chemistry, Spytag/Spycatcher, oxime ligation, condensation reactions. In some embodiments, the peptide epitope or protein antigen is covalently coupled to the self-assembling domain. In some embodiments, the peptide epitope or protein antigen is attached to the self-assembling domain through a thiol reactive group. The peptide epitope or protein antigen may be covalently coupled to a terminus of the self-assembling domain. In some embodiments, the peptide epitope or protein antigen is covalently coupled to the N-terminus of the self-assembling domain. In some embodiments, the peptide epitope or protein antigen is covalently coupled to the C-terminus of the self-assembling domain. The conjugation of the peptide epitope or protein antigen to a terminus of the self-assembling domain may orient the peptide epitopes or protein antigens towards the exterior of the supramolecular complexes or nanofibers detailed below. In some embodiments, the peptide epitopes or protein antigens are exposed on the exterior surface of the supramolecular complexes or nanofibers detailed below.

The peptide epitope or protein antigen may be linked to the N-terminal end or the C-terminal end of the self-assembling domain via a peptide linker.

### c. Peptide Linker

A linker may be between the peptide epitope or protein antigen and the self-assembling domain. In some embodiments, the peptide epitope or protein antigen is attached to the self-assembling domain through a thiol reactive group in the linker. The peptide linker comprises a polypeptide of 3 to 10 amino acids, or 3 to 25 amino acids. In some embodiments, the peptide linker comprises a polypeptide having an amino acid sequence selected from SEQ ID NO: 4 (SGSG), SEQ ID NO: 9 (Gₙ wherein n is an integer from 1 to 10), SEQ ID NO: 10 (SGSGₙ wherein n is an integer from 1 to 10), SEQ ID NO: 11 (GSGS), SEQ ID NO: 12 (SSSS), SEQ ID NO: 13 (GGGS), SEQ ID NO: 14 (GGC), SEQ ID NO: 15 (GGS), SEQ ID NO: 16 ((GGC)₈), SEQ ID NO: 17 (G₄S)₃, and SEQ ID NO: 18 (GGAAY). The peptide linker may be cleavable by a protease. In some embodiments, the peptide linker comprises a polypeptide having an amino acid sequence of SEQ ID NO: 4 (SGSG). In some embodiments, the conjugate includes more than one peptide linker. The peptide-polymer conjugate may include less than 20, less than 15, less than 10, or less than 5 peptide linkers. The peptide-polymer conjugate may include between 1 and 20, between 5 and 15, or between 1 and 5 peptide linkers. Multiple peptide linkers may be positioned adjacent to one another.

### d. Polyethylene Glycol (PEG) Domain

The peptide-polymer conjugate includes a polyethylene glycol (PEG) domain. The PEG domain includes at least one ethylene unit. The PEG domain is linked to the other of the N-terminal end and the C-terminal end of the self-assembling domain (from the peptide epitope or protein antigen). In some embodiments, the PEG domain is linked to the N-terminal end of the self-assembling domain. In some embodiments, the PEG domain is linked to the C-terminal end of the self-assembling domain. The PEG domain may have an average molecular weight of 300-5000 Da. The PEG domain may have an average molecular weight of at least about 300 Da, at least about 400 Da, at least about 500 Da, at least about 600 Da, at least about 700 Da, at least about 800 Da, at least about 900 Da, at least about 1000 Da, at least about 2000 Da, at least about 3000 Da, at least about 4000 Da, or at least about 5000 Da. The PEG domain may have an average molecular weight of less than about 5000 Da, less than about 4000 Da, less than about 3000 Da, less than about 2000 Da, or less than about 1000 Da. In some embodiments, the PEG domain has an average molecular weight of 350 Da (which may have a hydrodynamic radius of about 0.43 nm). In some embodiments, the PEG domain has an average molecular weight of 1000 Da (which may have a hydrodynamic radius of about 0.80 nm). In some embodiments, the PEG domain has an average molecular weight of 2000 Da (which may have a hydrodynamic radius of about 1.20 nm). In some embodiments, the PEG domain has an average molecular weight of 3000 Da (which may have a hydrodynamic radius of about 1.51 nm). The molecular weight may be calculated as the number average molecular weight. The PEG domain may be of the formula —(O—CH₂—CH₂)ₙ—OH, —(O—CH₂—CH₂)ₙ—O—C₁₋₄ alkyl, —(CH₂—CH₂—O)ₙ—CH₂—CH₂—OH, or —(CH₂—CH₂—O)ₙ—CH₂—CH₂—O—C₁₋₄ alkyl, wherein n is an integer between 1 and 200. In some embodiments, the PEG domain is of the formula —(O—CH₂—CH₂)ₙ—OH or —(O—CH₂-CH₂)ₙ—O—C₁₋₄ alkyl for N-terminal PEGylation. In some embodiments, the PEG domain is of the formula —(CH₂—CH₂—O)ₙ—CH₂—CH₂—OH or —(CH₂—CH₂—O)ₙ—CH₂—CH₂—O—C₁₋₄ alkyl for C-terminal PEGylation. In some embodiments, C₁₋₄ alkyl is methyl (for example, methoxy-terminated PEG, also referred to as mPEG). In some embodiments, C₁₋₄ alkyl is ethyl.

The PEG domain may be linked to the self-assembling domain by a linker. In some embodiments, the linker comprises a peptide linker as detailed above. In other embodiments, the linker comprises —NH₂—, —COO—, or any suitable linker known by those of skill in the art. Linkers and methods of PEGylation are described in, for example, Turecek et al. (Journal of Pharmaceutical Sciences 2016, 105, 460-475).

### e. Supramolecular Complex

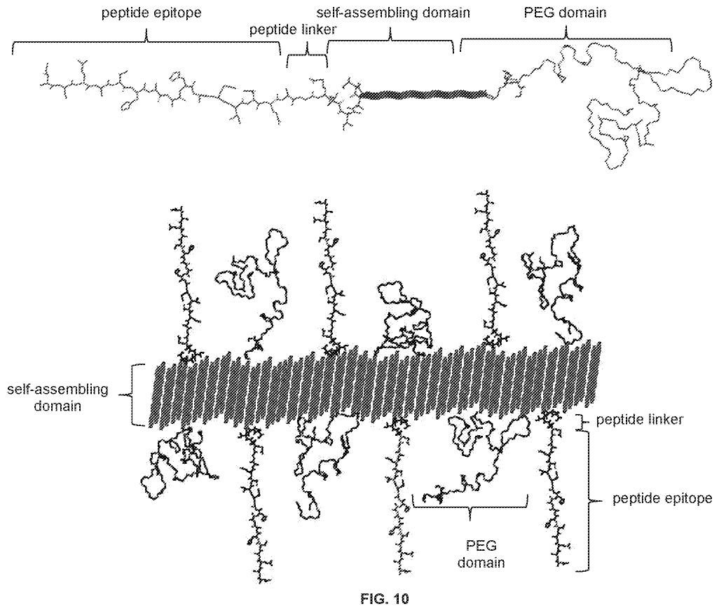

Further provided herein is a supramolecular complex comprising a plurality of the peptide-polymer conjugates detailed herein. A plurality of peptide-polymer conjugates may self-assemble into a supramolecular complex. The supramolecular complex may also be referred to as a nanofiber. In some embodiments, the plurality of peptide-polymer conjugates assemble with each other along an axis perpendicular to the length of each individual peptide-polymer conjugate, as shown, for example, in FIG. 10 (bottom). The plurality of peptide-polymer conjugates may assemble with each other along an axis perpendicular to the length of the self-assembling domain of each individual peptide-polymer conjugate. The conjugate may self-assemble into long nanofibers.

In some embodiments, each self-assembling domain of the supramolecular complex forms or comprises at least one alpha helix. In some embodiments, each self-assembling domain of the supramolecular complex forms or comprises at least one beta sheet. For example, Q11 peptide (SEQ ID NO: 1) forms beta-sheets, and OVAQ11-PEG (SEQ ID NO: 6) nanofibers have beta-sheet structure as well. In some embodiments, the supramolecular structure is coated on the exterior with a plurality of PEG domains. In some embodiments, the supramolecular structure is coated on the exterior with a plurality of peptide epitopes or protein antigens.

In some embodiments, the nanofibers have a length of 50 nm to 50,000 nm. The nanofibers may have uniform width. In some embodiments, the nanofibers have a width of 5-100 nm. The supramolecular complex may form in a variety of suitable compositions or solutions. The supramolecular complex may form in, for example, phosphate buffered saline (PBS) or other buffered salt solutions; cell culture medium; body fluids such as blood, serum, plasma, and interstitial fluid; or a combination thereof. The supramolecular complex may form at about pH 2-12, about pH 2-6, about pH 2-8, about pH 6-8, about pH 6-12, or about pH 8-12. In some embodiments, the supramolecular complex forms at physiological pH. In some embodiments, the supramolecular complex forms at about pH 6 to about pH 8.

In some embodiments, the peptide-polymer conjugates, or a supramolecular complex thereof, are non-toxic to a subject or to cells thereof.

The supramolecular complex may include the same peptide-polymer conjugates. As used herein, “same” may also be referred to as “identical.” The supramolecular complex may include a plurality of different peptide-polymer conjugates. In some embodiments of the supramolecular complex, the plurality of peptide-polymer conjugates comprises a plurality of non-identical peptide-polymer conjugates. In some embodiments of the supramolecular complex, the plurality of peptide-polymer conjugates comprises, consists of, or consists essentially of a plurality of identical peptide-polymer conjugates. In some embodiments, each peptide-polymer conjugate of a supramolecular complex is identical. In some embodiments, adjacent peptide-polymer conjugates in a supramolecular complex are identical. In some embodiments, each peptide-polymer conjugate of a supramolecular complex is different from each other. In some embodiments, adjacent peptide-polymer conjugates in a supramolecular complex are different from each other. The supramolecular complex may comprise at least, at most, or exactly 1, 2, 3, 4, 5, 6, 7, 8, 9, 10, 11, 12, 13, 14, 15, 16, 17, 18, 19, 20, 30, 40, 50, 100, 500, 1000, or 10,000 different peptide-polymer conjugates (or any derivable range therein). In some embodiments, the supramolecular complex includes n different peptide-polymer conjugates, wherein n is an integer from 1 to 10,000. The relative ratio of one peptide-polymer conjugate to another in the supramolecular complex may be at least, at most, or exactly 1, 2, 3, 4, 5, 6, 7, 8, 9, 10, 11, 12, 13, 14, 15, 16, 17, 18, 19, 20, 50, 100, 200, 300, 400, or 500 to 1, 2, 3, 4, 5, 6, 7, 8, 9, 10, 11, 12, 13, 14, 15, 16, 17, 18, 19, 20, 50, 100, 200, 300, 400, or 500 (or any derivable range therein).

The supramolecular complex may include the same or a plurality of different self-assembling domains. In some embodiments, each peptide-polymer conjugate of a supramolecular complex comprises the same self-assembling domain. In some embodiments, adjacent peptide-polymer conjugates in a supramolecular complex comprise the same self-assembling domain. In some embodiments, each peptide-polymer conjugate of a supramolecular complex comprises a different self-assembling domain. In some embodiments, adjacent peptide-polymer conjugates in a supramolecular complex comprise different self-assembling domains. The supramolecular complex may comprise at least, at most, or exactly 1, 2, 3, 4, 5, 6, 7, 8, 9, 10, 11, 12, 13, 14, 15, 16, 17, 18, 19, 20, 30, 40, 50, 100, 500, 1000, or 10,000 different self-assembling domains (or any derivable range therein). In some embodiments, the supramolecular complex includes n different self-assembling domains, wherein n is an integer from 1 to 10,000. The relative ratio of one self-assembling domain to another in the supramolecular complex may be at least, at most, or exactly 1, 2, 3, 4, 5, 6, 7, 8, 9, 10, 11, 12, 13, 14, 15, 16, 17, 18, 19, 20, 50, 100, 200, 300, 400, or 500 to 1, 2, 3, 4, 5, 6, 7, 8, 9, 10, 11, 12, 13, 14, 15, 16, 17, 18, 19, 20, 50, 100, 200, 300, 400, or 500 (or any derivable range therein).

The supramolecular complex may include the same or a plurality of different peptide epitopes or protein antigens. In some embodiments, each peptide-polymer conjugate of a supramolecular complex comprises the same peptide epitope or protein antigen. In some embodiments, adjacent peptide-polymer conjugates in a supramolecular complex comprise the same peptide epitope or protein antigen. In some embodiments, each peptide-polymer conjugate of a supramolecular complex comprises a different peptide epitope or protein antigen. In some embodiments, adjacent peptide-polymer conjugates in a supramolecular complex comprise different peptide epitopes or protein antigens. The supramolecular complex may comprise at least, at most, or exactly 1, 2, 3, 4, 5, 6, 7, 8, 9, 10, 11, 12, 13, 14, 15, 16, 17, 18, 19, 20, 30, 40, 50, 100, 500, 1000, or 10,000 different peptide epitopes or protein antigens (or any derivable range therein). In some embodiments, the supramolecular complex includes n different peptide epitopes or protein antigens, wherein n is an integer from 1 to 10,000. The relative ratio of one peptide epitope or protein antigen to another in the supramolecular complex may be at least, at most, or exactly 1, 2, 3, 4, 5, 6, 7, 8, 9, 10, 11, 12, 13, 14, 15, 16, 17, 18, 19, 20, 50, 100, 200, 300, 400, or 500 to 1, 2, 3, 4, 5, 6, 7, 8, 9, 10, 11, 12, 13, 14, 15, 16, 17, 18, 19, 20, 50, 100, 200, 300, 400, or 500 (or any derivable range therein).

The supramolecular complex may include the same or a plurality of different PEG domains. In some embodiments, each peptide-polymer conjugate of a supramolecular complex comprises the same PEG domain. In some embodiments, adjacent peptide-polymer conjugates in a supramolecular complex comprise the same PEG domain. In some embodiments, each peptide-polymer conjugate of a supramolecular complex comprises a different PEG domain. In some embodiments, adjacent peptide-polymer conjugates in a supramolecular complex comprise different PEG domains. The supramolecular complex may comprise at least, at most, or exactly 1, 2, 3, 4, 5, 6, 7, 8, 9, 10, 11, 12, 13, 14, 15, 16, 17, 18, 19, 20, 30, 40, 50, 100, 500, 1000, or 10,000 different PEG domains (or any derivable range therein). In some embodiments, the supramolecular complex includes n different PEG domains, wherein n is an integer from 1 to 10,000. The relative ratio of one PEG domain to another in the supramolecular complex may be at least, at most, or exactly 1, 2, 3, 4, 5, 6, 7, 8, 9, 10, 11, 12, 13, 14, 15, 16, 17, 18, 19, 20, 50, 100, 200, 300, 400, or 500 to 1, 2, 3, 4, 5, 6, 7, 8, 9, 10, 11, 12, 13, 14, 15, 16, 17, 18, 19, 20, 50, 100, 200, 300, 400, or 500 (or any derivable range therein)

In some embodiments, the supramolecular complex includes at least one peptide-polymer conjugate without a peptide epitope or protein antigen, or at least one peptide-polymer conjugate without a PEG domain, or at least one peptide-polymer conjugate without any of a peptide epitope or protein antigen or PEG domain, or a combination thereof.

### f. Synthesis of Peptides and Proteins

The peptides described herein, such as the self-assembling domain, the peptide epitope, and/or the peptide linker, can be chemically synthesized using standard chemical synthesis techniques. In some embodiments the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides described herein. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85: 2149-2156, and Stewart et al. (1984) Solid Phase Peptide Synthesis, 2nd ed. Pierce Chem. Co., Rockford, Ill. In some embodiments, the self-assembling peptide is synthesized by a solid phase peptide synthesis.

The proteins described herein, such as the protein antigen, may be produced recombinantly according to techniques known to those of skill in the art.

### 3. Immune Response and Immunoassays

As discussed above, the peptide-polymer conjugates, or a supramolecular complex thereof, provided herein may evoke or induce an immune response in a subject against an antigen or epitope. To induce an immune response, the peptide-polymer conjugates, or a supramolecular complex thereof, may be taken up by an antigen presenting cell (APC), processed, and presented on a major histocompatibilty complex (MHC). In some embodiments, the immune response can protect against or treat a subject having, suspected of having, or at risk of developing an infection or related disease, or a pathological condition such as cancer or autoimmunity. Other uses of the peptide-polymer conjugates, or a supramolecular complex thereof, may be to provide effective vaccines, such as cancer vaccines. The peptide-polymer conjugates, or a supramolecular complex thereof, detailed herein may induce an immune response. The immune response may be an antigen-specific immune response. Administration of the peptide-polymer conjugates, or a supramolecular complex thereof, may raise antibodies specific to the peptide epitope or protein antigen thereof. In some embodiments, the immune response includes or favors a Th2 response. In some embodiments, the antigen-specific immune response is temporary or not life-long. In some embodiments, the immune response comprises IgG1 antibody isotypes. In some embodiments, the immune response is an anti-cancer immune response. The peptide-polymer conjugates, or a supramolecular complex thereof, may have increased immunogenicity relative to a control. In some embodiments, the control comprises a peptide epitope or protein antigen without a self-assembling domain. In some embodiments, the control comprises a peptide epitope or protein antigen without a self-assembling domain or a PEG domain. In some embodiments, the control comprises a peptide epitope or protein antigen without a PEG domain.

Further provided herein is the implementation of serological assays to evaluate whether and to what extent an immune response is induced or evoked by the peptide-polymer conjugates, or a supramolecular complex thereof. There are many types of immunoassays that can be implemented. Immunoassays include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. Certain immunoassays are the various types of enzyme linked immunosorbent assays (ELISAs) and radioimmunoassays (RIA) known in the art. Immunohistochemical detection using tissue sections is also useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody or epitope, one may generally incubate the wells of the plate with a solution of the antigen or antibody or epitope, either overnight or for a specified period. The wells of the plate will then be washed to remove incompletely-adsorbed material. Any remaining available surfaces of the wells may then be “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

### 4. Protective Immunity

In some embodiments, the peptide-polymer conjugates, or a supramolecular complex thereof, may confer protective immunity to a subject. Protective immunity refers to a subject's ability to mount a specific immune response that protects the subject from developing a particular disease or condition that involves the agent against which there is an immune response. An immunogenically effective amount is capable of conferring protective immunity to the subject.

Different polypeptides as detailed herein may have different functionalities. While according to one aspect, a polypeptide is derived from an antigen or immunogen designed to induce an active immune response in a recipient, according to another aspect, a polypeptide is derived from an antibody which results following the elicitation of an active immune response in, for example, an animal, and which can serve to induce a passive immune response in the recipient.

The phrase “immune response” or “immunological response” refers to the development of a humoral (antibody mediated), cellular (mediated by antigen-specific T cells or their secretion products), or both humoral and cellular response directed against an antigen, epitope, protein, peptide, carbohydrate, or polypeptide in a recipient patient. Such a response can be an active response induced by administration of an epitope or antigen or immunogen or a passive response induced by administration of antibody, antibody containing material, or primed T-cells. A cellular immune response may be elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an epitope or antigen.

As used herein “passive immunity” refers to any immunity conferred upon a subject without administration of an epitope or antigen to the subject. “Passive immunity” therefore includes, but is not limited to, administration of activated immune effectors including cellular mediators or protein mediators (e.g., monoclonal and/or polyclonal antibodies) of an immune response. A monoclonal or polyclonal antibody composition may be used in passive immunization for the prevention or treatment of infection by organisms that carry the antigen recognized by the antibody. An antibody composition may include antibodies that bind to a variety of epitopes or antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as gram-positive bacteria, gram-negative bacteria, including but not limited to staphylococcus bacteria.

Passive immunity may be imparted to a patient or subject by administering to the patient immunoglobulins (Ig) and/or other immune factors obtained from a donor or other non-patient source having a known immunoreactivity. In other aspects, an antigenic composition as detailed herein can be administered to a subject who then acts as a source or donor for globulin, produced in response to challenge with the antigenic composition (“hyperimmune globulin”), that contains antibodies directed against, for example, Staphylococcus or other organism. A subject thus treated would donate plasma from which hyperimmune globulin would then be obtained, via conventional plasma-fractionation methodology, and administered to another subject in order to impart resistance against or to treat Staphylococcus infection. Hyperimmune globulins are particularly useful for immune-compromised individuals, for individuals undergoing invasive procedures or where time does not permit the individual to produce their own antibodies in response to vaccination. See U.S. Pat. Nos. 6,936,258, 6,770,278, 6,756,361, 5,548,066, 5,512,282, 4,338,298, and 4,748,018, each of which is incorporated herein by reference in its entirety, for exemplary methods and compositions related to passive immunity.

For purposes of this specification and the accompanying claims the terms “epitope” and “antigenic determinant” may be used interchangeably to refer to a site on an antigen to which B and/or T cells respond or recognize. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by 3H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

As used herein and in the claims, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins.

Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains.

In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies can be produced by hyperimmunization of an appropriate donor with the antigen or ex-vivo by use of primary cultures of splenic cells or cell lines derived from spleen (Anavi, 1998; Huston et al., 1991; Johnson et al., 1991; Mernaugh et al., 1995).

As used herein and in the claims, the phrase “an immunological portion of an antibody” includes a Fab fragment of an antibody, a Fv fragment of an antibody, a heavy chain of an antibody, a light chain of an antibody, a heterodimer consisting of a heavy chain and a light chain of an antibody, a variable fragment of a light chain of an antibody, a variable fragment of a heavy chain of an antibody, and a single chain variant of an antibody, which is also known as scFv. In addition, the term includes chimeric immunoglobulins which are the expression products of fused genes derived from different species, one of the species can be a human, in which case a chimeric immunoglobulin is said to be humanized. Typically, an immunological portion of an antibody competes with the intact antibody from which it was derived for specific binding to an antigen.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

### 5. Pharmaceutical Compositions

The peptide-polymer conjugate as detailed herein, or a plurality or a complex thereof, may be formulated into pharmaceutical compositions accordance with standard techniques well known to those skilled in the pharmaceutical art. The composition may comprise the peptide-polymer conjugate, or a plurality thereof, and a pharmaceutically acceptable carrier. The term “pharmaceutically acceptable carrier,” as used herein, means a non-toxic, inert solid, semi-solid or liquid filler, diluent, encapsulating material or formulation auxiliary of any type.

Further provided are methods for making the compositions as detailed herein comprising mixing a plurality of the peptide-polymer conjugates and a carrier to make a supramolecular complex.

The route by which the disclosed peptide-polymer conjugates are administered and the form of the composition will dictate the type of carrier to be used. The pharmaceutical composition may be in a variety of forms, suitable, for example, for systemic administration (e.g., oral, rectal, sublingual, buccal, implants, intranasal, intravaginal, transdermal, intravenous, intraarterial, intratumoral, intraperitoneal, or parenteral) or topical administration (e.g., dermal, pulmonary, nasal, aural, ocular, liposome delivery systems, or iontophoresis). In some embodiments, the pharmaceutical composition is for administration to a subject's mucosal surfaces. Techniques and formulations may generally be found in “Remington's Pharmaceutical Sciences,” (Meade Publishing Co., Easton, Pa.). Pharmaceutical compositions must typically be sterile and stable under the conditions of manufacture and storage. All carriers are optional in the compositions.

Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof.

Suitable diluents include, for example, sugars such as glucose, lactose, dextrose, and sucrose; diols such as propylene glycol; calcium carbonate; sodium carbonate; sugar alcohols, such as glycerin; mannitol; sorbitol; cellulose; starch; and gelatin. The amount of diluent(s) in a systemic or topical composition may typically be about 50 to about 90%.

Suitable lubricants include, for example, silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil, and oil of theobroma. The amount of lubricant(s) in a systemic or topical composition may typically be about 5 to about 10%.

Suitable binders include, for example, polyvinyl pyrrolidone; magnesium aluminum silicate; starches such as corn starch and potato starch; gelatin; tragacanth; sucrose; and cellulose and its derivatives, such as sodium carboxymethylcellulose, ethyl cellulose, methylcellulose, microcrystalline cellulose, and hydroxypropyl methylcellulose. The amount of binder(s) in a systemic composition may typically be about 5 to about 50%.

Suitable disintegrants include, for example, agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition may typically be about 0.1 to about 10%.

Suitable colorants include, for example, a colorant such as an FD&C dye. When used, the amount of colorant in a systemic or topical composition may typically be about 0.005 to about 0.1%.

Suitable flavors include, for example, menthol, peppermint, and fruit flavors. The amount of flavor(s), when used, in a systemic or topical composition may typically be about 0.1 to about 1.0%.

Suitable sweeteners include, for example, aspartame and saccharin, or a combination thereof. The amount of sweetener(s) in a systemic or topical composition may typically be about 0.001 to about 1%.

Suitable antioxidants include, for example, butylated hydroxyanisole (“BHA”), butylated hydroxytoluene (“BHT”), and vitamin E. The amount of antioxidant(s) in a systemic or topical composition may typically be about 0.1 to about 5%.

Suitable preservatives include, for example, benzalkonium chloride, methyl paraben, and sodium benzoate. The amount of preservative(s) in a systemic or topical composition may typically be about 0.01 to about 5%.

Suitable glidants include, for example, silicon dioxide. The amount of glidant(s) in a systemic or topical composition may typically be about 1 to about 5%.

Suitable solvents include, for example, water, isotonic saline, ethyl oleate, glycerine, castor oils, hydroxylated castor oils, alcohols such as ethanol or isopropanol, methylene chloride, ethylene glycol monoethyl ether, diethylene glycol monobutyl ether, diethylene glycol monoethyl ether, dimethylsulfoxide, dimethyl formamide, tetrahydrofuran, and phosphate buffer solutions, and combinations thereof. The amount of solvent(s) in a systemic or topical composition is typically from about 0 to about 100%, or 0% to about 95%.

Suitable suspending agents include, for example, AVICEL RC-591 (from FMC Corporation of Philadelphia, PA) and sodium alginate. The amount of suspending agent(s) in a systemic or topical composition may typically be about 1 to about 8%.

Suitable surfactants include, for example, lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp.587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition may typically be about 0.1% to about 5%.

Suitable emollients include, for example, stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition may typically be about 5% to about 95%.

Suitable propellants include, for example, propane, butane, isobutane, dimethyl ether, carbon dioxide, nitrous oxide, and combinations thereof. The amount of propellant in a topical composition may be about 0% to about 95%.

Suitable humectants include, for example, glycerin, sorbitol, sodium 2-pyrrolidone-5-carboxylate, soluble collagen, dibutyl phthalate, gelatin, and combinations thereof. The amount of humectant in a topical composition may be about 0% to about 95%.

Suitable powders include, for example, beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition may typically be 0% to 95%.

Suitable pH adjusting additives include, for example, HCI or NaOH in amounts sufficient to adjust the pH of a topical pharmaceutical composition.

In some embodiments, the pharmaceutically acceptable carrier is a sugar such as lactose, glucose, and sucrose. In some embodiments, the pharmaceutically acceptable carrier is a starch such as, for example, corn starch and potato starch. In some embodiments, the pharmaceutically acceptable carrier is cellulose and its derivatives such as, but not limited to, sodium carboxymethyl cellulose, ethyl cellulose and cellulose acetate. In some embodiments, the pharmaceutically acceptable carrier is powdered tragacanth, malt, gelatin, or talc. In some embodiments, the pharmaceutically acceptable carrier is an excipient such as, but not limited to, cocoa butter and suppository waxes. In some embodiments, the pharmaceutically acceptable carrier is oil such as, but not limited to, peanut oil, cottonseed oil, safflower oil, sesame oil, olive oil, corn oil and soybean oil. In some embodiments, the pharmaceutically acceptable carrier is a glycol, such as propylene glycol. In some embodiments, the pharmaceutically acceptable carrier is an ester such as, but not limited to, ethyl oleate and ethyl laurate. In some embodiments, the pharmaceutically acceptable carrier is an agar. In some embodiments, the pharmaceutically acceptable carrier is a buffering agent such as, but not limited to, magnesium hydroxide and aluminum hydroxide. In some embodiments, the pharmaceutically acceptable carrier is alginic acid, pyrogen-free water, isotonic saline, Ringer's solution, ethyl alcohol, or a phosphate buffer solution. In some embodiments, the pharmaceutically acceptable carrier is a non-toxic compatible lubricant such as, but not limited to, sodium lauryl sulfate and magnesium stearate.

Compositions for oral administration can have various dosage forms. For example, solid forms include tablets, capsules, granules, and bulk powders. Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Capsules (including implants, time release, and sustained release formulations) typically include a compound, and a carrier including one or more diluents disclosed above in a capsule comprising gelatin. Granules typically comprise a compound, and preferably glidants such as silicon dioxide to improve flow characteristics. Implants can be of the biodegradable or the non-biodegradable type.

Compositions for oral administration can have solid forms. Solid oral compositions may be coated by conventional methods, typically with pH or time-dependent coatings, such that a compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes, and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Compositions for topical administration can be applied locally to the skin and may be in any form including solids, solutions, oils, creams, ointments, gels, lotions, shampoos, leave-on and rinse-out hair conditioners, milks, cleansers, moisturizers, sprays, skin patches, and the like. The carrier of the topical composition preferably aids penetration of the compound into the skin. In the topical compositions, the carrier includes a topical carrier. Suitable topical carriers can include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications may include propylene glycol, dimethyl isosorbide, and water, and even more particularly, phosphate buffered saline, isotonic water, deionized water, monofunctional alcohols, and symmetrical alcohols. The carrier of a topical composition may further include one or more ingredients selected from emollients, propellants, solvents, humectants, thickeners, powders, fragrances, pigments, and preservatives, all of which are optional.

Although the amounts of components in the compositions may vary depending on the type of composition prepared, in general, systemic compositions may include 0.01% to 50% of a peptide-polymer conjugate and 50% to 99.99% of one or more carriers. Compositions for parenteral administration may typically include 0.1% to 10% of a peptide-polymer conjugate and 90% to 99.9% of one or more carriers. Oral dosage forms may include, for example, at least about 5%, or about 25% to about 50% of a peptide-polymer conjugate. The oral dosage compositions may include about 50% to about 95% of carriers, or from about 50% to about 75% of carriers. The amount of the carrier employed in conjunction with a disclosed peptide-polymer conjugate is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

### 6. Administration

The peptide-polymer conjugates or complexes as detailed herein, or the pharmaceutical compositions comprising the same, may be administered to a subject. A composition may comprise the peptide-polymer conjugate. The peptide-polymer conjugate can be formulated into a composition and administered in dosages and by techniques well known to those skilled in the medical arts taking into consideration such factors as the age, sex, weight, and condition of the particular subject, and the route of administration.

The peptide-polymer conjugate can be administered prophylactically or therapeutically. In prophylactic administration, the peptide-polymer conjugate can be administered in an amount sufficient to induce a response. In therapeutic applications, the peptide-polymer conjugates are administered to a subject in need thereof in an amount sufficient to elicit a therapeutic effect. The peptide-polymer conjugate may be administered in a therapeutically effective amount.

For example, a therapeutically effective amount of a peptide-polymer conjugate, complex, or composition thereof, may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The peptide-polymer conjugate can be administered by methods well known in the art as described in Donnelly et al. (Ann. Rev. Immunol. 1997, 15, 617-648); Feigner et al. (U.S. Pat. No. 5,580,859, issued Dec. 3, 1996); Feigner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The peptide-polymer conjugate or complex thereof can be complexed to particles or beads that can be administered to an individual, for example, using a vaccine gun. One skilled in the art would know that the choice of a pharmaceutically acceptable carrier, including a physiologically acceptable compound, depends, for example, on the route of administration.

The peptide-polymer conjugate can be delivered via a variety of routes. Typical delivery routes include parenteral administration, e.g., intradermal, intramuscular or subcutaneous delivery. Other routes include oral administration, sublingual, intranasal, intravaginal, transdermal, intravenous, intraarterial, intratumoral, intrathecal, intraperitoneal, rectal, intravaginally, and epidermal routes. In some embodiments, the peptide-polymer conjugate is administered sublingually, intravaginally, intravenously, intraarterially, or intraperitoneally to the subject.

The peptide-polymer conjugate can be a liquid preparation such as a suspension, syrup, or elixir. The peptide-polymer conjugate can be incorporated into liposomes, microspheres, or other polymer matrices (such as by a method described in Feigner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The peptide-polymer conjugate may be used as a vaccine. The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation can be carried out via a minimally invasive device.

In some embodiments, the peptide-polymer conjugate is administered in a controlled release formulation. The peptide-polymer conjugate may be released into the circulation, for example. In some embodiments, the peptide-polymer conjugate may be released over a period of at least about 1 day, at least about 2 days, at least about 3 days, at least about 4 days, at least about 5 days, at least about 6 days, at least about 7 days, at least about 1 week, at least about 1.5 weeks, at least about 2 weeks, at least about 2.5 weeks, at least about 3.5 weeks, at least about 4 weeks, or at least about 1 month.

Upon or shortly after administration, the peptide-polymer conjugate may elicit an antibody or immune response to the peptide epitope. In some embodiments, the peptide-polymer conjugate elicits a humoral immune responses. In some embodiments, the peptide-polymer conjugate elicits a cellular immune response In some embodiments, the peptide-polymer conjugate elicits humoral and cellular immune responses. In some embodiments, the peptide-polymer conjugate elicits a T cell response.

In some embodiments, the peptide-polymer conjugate is co-administered with an adjuvant. In some embodiments, the peptide-polymer conjugate is not co-administered with an adjuvant. Adjuvants may include, for example, cholera toxin, CpG, cyclic-di-GMP, cyclic-di-AMP, or a combination thereof. Cholera toxin may include, for example, Cholera Toxin B.

### a. Sublingual Administration

In some embodiments, the peptide-polymer conjugate or complex thereof is delivered sublingually. In addition to those detailed above, compositions and carriers that may be suitable for sublingual administration are described in U.S. Pat. No. 5,487,898 and U.S. Patent Application Publication No. 2003/0165512, which are incorporated herein by reference. Sublingual administration is through the mucosa of the floor of the mouth and ventral side of the tongue. Mucosal vaccines, which are vaccines delivered across the mucosal barriers through which most pathogens enter the body, may have key advantages over vaccines delivered through systemic injection via needles. Immunologically, these vaccines can elicit protective antibodies in mucosal compartments to neutralize and clear pathogens from the body before they take up residence. Furthermore, depending on the chosen mucosal delivery route, these vaccines may have significant logistical and financial benefits.

Sublingual vaccination may elicit antibody responses in an anatomically broad range of mucosal surfaces, such as the upper and lower respiratory tracts and the reproductive tract, in addition to raising systemic responses in the blood. In some embodiments, sublingual vaccination may elicit antibody responses in genital tracts. Sublingual vaccination may also be a favorable delivery route to encourage compliance, as it does not require needles, thereby allowing for pain-free and potentially self-administered vaccination using dissolvable tablets or wafers. Because it does not require needles or trained personnel, sublingual immunization can be done at a lower cost, which may be constraints when designing vaccines for developing nations. Sublingual administration may also reduce or prevent the reuse of needles, and thereby prevent or reduce infections.

Upon sublingual administration, the peptide-polymer conjugate may penetrate through the salivary mucus layer and be sampled by dendritic cells at the sublingual epithelium. Mucus is composed largely of mucins, which are glycoprotein fibers that crosslink and entangle to form a network that may restrict the movement of pathogens through mucus barriers to promote clearance from the body. Without being limited to theory, it may be that the PEG domain of the peptide-polymer conjugate detailed herein may reduce or prevent interactions with the mucus network, thereby minimizing mucosal-adhesion and promoting diffusion. The PEG domain may reduce binding of mucin to the peptide-polymer conjugates. Mucin binding to the peptide-polymer conjugates may be negatively correlated with the molecular weight of the PEG domain. Mucin binding affinity may not be correlated with the molecular weight of the PEG domain.

### 7. Methods

### a. Methods of Immunizing a Subject

Provided here are methods of immunizing a subject. The method may include administering to the subject an effective amount of the peptide-polymer conjugate or the supramolecular complex thereof, or a composition comprising the same, as detailed herein. In some embodiments, the peptide-polymer conjugate or the supramolecular complex thereof, or a composition comprising the same, is administered sublingually. In some embodiments, the peptide-polymer conjugate or the supramolecular complex thereof, or a composition comprising the same, is co-administered with an adjuvant.

Further provided is an antibody produced by an immunized subject as detailed herein.

### b. Methods of Eliciting an Immune Response

Provided here are methods of eliciting an immune response to a peptide epitope in a subject. The method may include administering to the subject an effective amount of the peptide-polymer conjugate or the supramolecular complex thereof, or a composition comprising the same, as detailed herein. In some embodiments, the peptide-polymer conjugate or the supramolecular complex thereof, or a composition comprising the same, is administered sublingually. In some embodiments, the peptide-polymer conjugate or the supramolecular complex thereof, or a composition comprising the same, is co-administered with an adjuvant.

In some embodiments, the immune response is an antigen-specific immune response. In some embodiments, the antigen-specific immunity is temporary and/or not life-long. Antigen-specific immunity refers to an adaptive immune response that occurs upon subsequent encounter with an antigenic determinant. In life-long immunity, vaccination protects the subject from environmental encounters with the antigen by inducing an immune response after the antigen has been encountered. In some embodiments, the immunity is temporary or lasts less than 20, 15, 10, 9, 8, 7, 6, 5, 4, 3, 2, or 1 years (or any derivable range therein). In some embodiments, the immune response comprises IgG1 antibody isotypes. In some embodiments, IgG1 antibody isotypes are the dominant antibody isotype produced in the immune response. In some embodiments, IgG1 antibody isotypes are significantly more in relation to the other antibody isotypes in the immune response. In some embodiments, the titer of IgG1 is at least 1, 1.5, 2, 2.5, or 3 log10 units higher than other isotypes.

Further provided is an antibody produced in the immune response of the methods as detailed herein.

### 8. Examples

### EXAMPLE 1

### Materials and Methods

Peptide Synthesis and Purification. OVAQ11 (H₂N-ISQAVHAAHAEINEAGR-SGSGQQKFQFQFEQQ-Am) and pOVA (H₂N-ISQAVHAAHAEINEAGR-Am) were synthesized using standard Fmoc solid-phase synthesis. For synthesis of OVAQ11-PEG and pOVA-PEG, peptides were synthesized using a PAP Tentagel resin, which leaves C-terminal 3000 MW PEG block after cleavage. Peptides were cleaved for 2 hours at room temperature in a 95/2.5/2.5 TFA/triisopropylsilane/water cocktail, followed by washing with cold diethyl ether. Peptides were purified by reverse-phase HPLC using either a C18 column (OVAQ11, pOVA, W-Q11) ora C4 column (OVAQ11-PEG, pOVA-PEG) and lyophilized. Peptide identity was confirmed using matrix-assisted laser desorption/ionization mass spectrometry on a Bruker Autoflex Speed LRF MALDI-TOF spectrometer using α-cyano-4-hydroxycinnamic acid as the matrix.

To prepare immunization formulations, lyophilized peptide was first dissolved at 8 mM in sterile water and incubated at 4° C. overnight. The solutions were then brought to the final concentration in 1X PBS by addition of sterile water and sterile 10X PBS and incubated at room temperature for 3 h. After incubation, sheared solutions were prepared by repeated passage through a 100 nm polycarbonate track-etched (PCTE) membrane using an Avanti Polar Lipids Mini-Extruder. For adjuvanted formulations, formulations included 2 μg of cholera toxin or 10 μg of CTB, which were added just prior to immunization.

Transmission Electron Microscopy (TEM). Peptides were prepared at 2 mM and diluted with 1X PBS to 0.2 mM, then 5 μL of peptide solution was deposited onto Formvar/carbon-coated 400 mesh copper grids. The sample was allowed to incubate for 1 min, washed with ultrapure water, and negatively stained for 1 min with 1% w/v uranyl acetate in water prior to wicking away with filter paper. Samples were imaged on an FEI Tecnai G² Twin electron microscope. Nanofiber lengths were measured using ImageJ.

Secondary Structure Analysis Using Circular Dichroism and Fluorescent Probes. For CD, peptides were prepared at 3 mM in 1x PBS, and diluted to 0.1 mM in potassium fluoride (KF) buffer just prior to analysis. CD spectra were collected on a Chiroscan Plus spectrometer from 220 nm to 260 nm in a 0.1 cm path length quartz cuvette.

For Thioflavin T (ThT) and pyrene assays, lyophilized peptides were dissolved in either a 4.0×10⁻³ wt % solution of ThT in 1X PBS or a 1.3×10⁻⁶ wt % solution of pyrene in 1×PBS and incubated at room temperature for 3 hours. A 100 μL aliquot of each solution was added to a black 96-well plate and read using a Molecular Devices Spectramax M2. For ThT, the excitation wavelength was 440 nm, and the emission wavelength was 488 nm. For pyrene, the excitation wavelength was 339 nm, and the emission wavelength was 373 nm.

Zeta-potential Measurements. Peptides were prepared at 2 mM and diluted to 0.2 mM with 1X PBS just prior to analysis. Zeta-potential was measured at 25° C. using a Malvern Nano ZetaSizer.

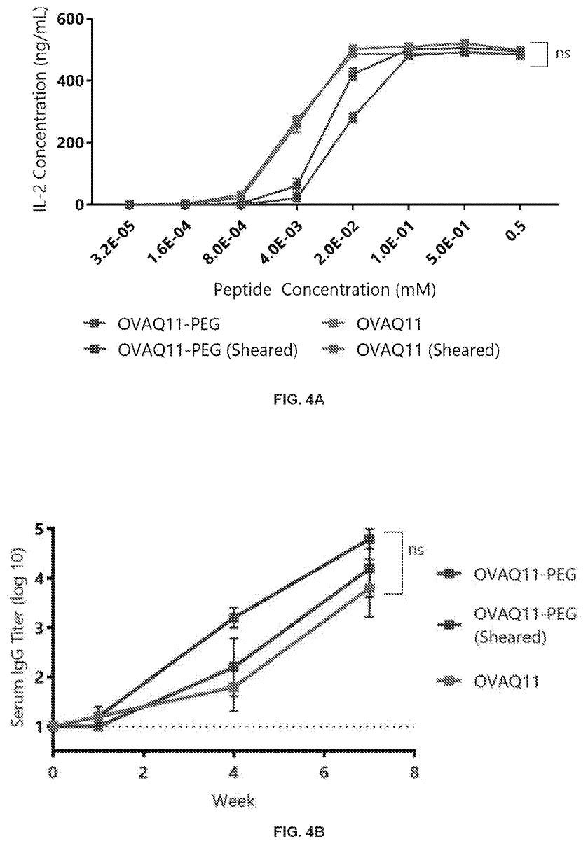

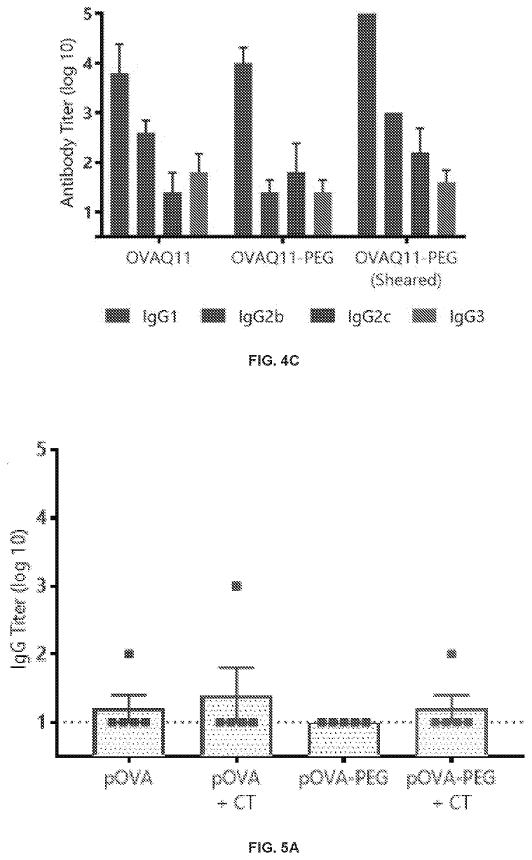

Mice and Immunizations. Female C57BL/6 mice were purchased from Envigo. Animal experiments were approved by the Institutional Care and Use Committees of Duke University and the University of Chicago for work at each respective location. Subcutaneous immunizations (FIG. 4A, FIG. 4B, FIG. 4C) were performed at the University of Chicago; all other mouse experiments were performed at Duke University. For subcutaneous immunizations, mice were anesthetized using isoflurane and given 2×50 μL injections of 2 mM peptide, one behind each shoulder, and booster immunizations of the same dose were given 4 weeks after the primary immunizations. For sublingual immunizations, mice were deeply anesthetized by a 100 μL intraperitoneal injection of a cocktail delivering 100 mg/kg ketamine and 10 mg/kg xylazine. A micropipette with a 20 μL tip was used to apply 7 μL of solution below the tongue, and the mice's heads were placed in anteflexion for 20 minutes following administration to prevent swallowing of the material.

In Vitro Presentation Assay. Bone marrow derived dendritic cells (BMDCs) were isolated from the femur and tibia of naïve mice and added at 2×10⁶/mL (5 mL) in complete RPMI medium to a 6-well plate. To each well, 1 pg of Flt-3L ligand was added, and the BMDCs were incubated at 37° C. for 8 d. The BMDCs were added at 2×10⁵/mL (50 μL) to a 96-well plate along with 50 μL of serially diluted peptides, and incubated at 37° C. for 2 h. The plates were centrifuged at 500× g for 5 min, the supernatant was aspirated, and 100 μL of DOBW cells resuspended at 5×10⁻⁵ cells/mL in complete DMEM media were added to each well and incubated overnight at 37 ° C. DOBW cells produce IL-2 when they encounter pOVA presented in MHC class II (I-A^{b}). The plates were centrifuged for 5 min at 500× g, the supernatant was collected, and the IL-2 concentration was measured using an ELISA kit (BD Bioscience, Franklin Lakes, NJ; Cat# 55148).

Collection of Serum and Mucosal Secretions. Serum was collected via the submandibular vein and stored at -80° C. prior to analysis. To collect vaginal washes, mice were anesthetized with isoflurane and the vagina was flushed with 40 μL of 1X PBS using a micropipette. Nasal wash was collected in post-mortem mice by flushing 200 μL of 1X PBS through the choanae. Mucosal samples were flash-frozen with dry ice and then stored at - 20° C. prior to analysis.

Measurement of Antibody Responses Using ELISA. For analysis of antigen- specific IgG by ELISA, plates were coated with a 20 μg/mL streptavidin solution and incubated overnight at 4° C. Plates were washed with 0.05% (w/v) Tween-20 in PBS (1X PBST), blocked by Superblock blocking buffer solution (Thermo Fisher Scientific, Waltham, MA; Cat #37515), washed again, and coated with 20 μg/mL of biotinylated pOVA for 1 h at room temperature. Sera or mucosal secretions were diluted in 1% (w/v) BSA in 1x PBST and added to the plate, and OVA-specific IgG was detected by horseradish peroxidase conjugated Fcγ fragment specific goat anti-mouse IgG (Jackson Immuno Research, West Grove, PA; Cat #115-035-071).

Measurement of T Cell Responses Using ELISPOT. To analyze T cell activation by ELISPOT, mice were sacrificed 7 d after the final booster immunization and spleens were harvested. Briefly, 0.25 million splenocytes in 200 μL were plated in each well of a 96-well ELISPOT plate (MilliporeSigma, Burlington, MA; MSIPS4510). The cells were stimulated with 5 μM pOVA peptide, left untreated as negative controls, or stimulated with Concanavalin A (ConA) as a positive controls. To detect IFNγ secreting cell spots, a biotinylated anti-mouse IFNγ (551881) detection antibody pair from BD Bioscience (Franklin Lakes, NJ), streptavidin-alkaline phosphatase (3310-0) from Mabtech (Nacka Strand, Sweden), and Sigmafast BCIP/NBT (Sigma, B5655) from Sigma Aldrich (St. Louis, MO) were used. Plates were imaged and counted by Zellnet Consulting using a Zeiss KS ELISPOT reader.

Statistical Analysis. Statistical analysis was performed as indicated in figure legends using GraphPad Prism and JMP Pro software. Means ±standard error of the mean (SEM) are presented.

### EXAMPLE 2

### Design and Characterization of Epitope-Bearing Peptide-Polymer Conjugates

We based our design fora sublingual peptide vaccine on the Q11 platform, which has been shown previously to elicit immune responses via the subcutaneous (Rudra JS, et al. PNAS 2010, 107, 622-627), intraperitoneal (Chen J, et al. Biomaterials 2013, 34, 8776-8785), and intranasal (Si Y, et al. Journal of Controlled Release 2018) routes. We hypothesized that promoting the transport of Q11 nanofibers through the salivary mucus layer would allow the nanofibers to be sampled by dendritic cells at the sublingual epithelium, initiating immune responses. Mucus is composed largely of mucins, glycoprotein fibers that crosslink and entangle to form a network that restricts the movement of pathogens through mucus barriers to promote clearance from the body. PEGylation may be an effective means of imparting mucus-penetrating properties to biomaterials. We sought to apply this concept to Q11 to create a mucus-penetrating nanofiber for sublingual vaccination. Sequences of the peptides are shown in TABLE 2, with a description of the parts of OVAQ11 (SEQ ID NO: 5) and OVAQ11-PEG (SEQ ID NO: 6) in TABLE 3.

TABLE 2
Sequences of the peptides.

| Peptide | Sequence |
|---|---|
| OVAQ11 | H₂N-ISQAVHAAHAEINEAGRSGSGQQKFQFQFEQQ- |
| | CONH₂ |
| | (SEQ ID NO: 5) |
| OVAQ11- | H₂N-ISQAVHAAHAEINEAGRSGSGQQKFQFQFEQQ- |
| PEG | NH-(CH₂-CH₂-O)ₙ-CH₂-CH₂-OH |
| | (SEQ ID NO: 6) |
| pOVA | H₂N-ISQAVHAAHAEINEAGR-CONH₂ |
| | (SEQ ID NO: 3) |
| pOVA-PEG | H₂N-ISQAVHAAHAEINEAGR-NH-(CH₂-CH₂-O)ₙ- |
| | CH₂-CH₂-OH |
| | (SEQ ID NO: 7) |
| Biotin-pOVA | Biotin-SGSG-ISQAVHAAHAEINEAGR-CONH₂ |
| | (SEQ ID NO: 8) |
| W-Q11 | H₂N-WSGSGQQKFQFQFEQQ-CONH₂ |
| | (SEQ ID NO: 2) |

TABLE 3
Parts of OVAQ11 and OVAQ11-PEG.

| | |
|---|---|
| | MW |
| Peptide | (Da) |

OVAQ11 (SEQ ID NO: 5)

| | | | | | |
|---|---|---|---|---|---|
| H₂N | ISQAVHAAHAEINEAGR | SGSG | QQKFQFQFEQQ | CONH₂ | 3528 |
| | OVA₃₂₃₋₃₃₉ epitope | peptide | self-assembling | | |
| | | linker | domain | | |

OVAQ11-PEG (SEQ ID NO: 6):

| | | | | | | |
|---|---|---|---|---|---|---|
| H₂N | ISQAVHAAHAEINEAGR | SGSG | QQKFQFQFEQQ | —NH— | PEG* | 6722 |
| | OVA₃₂₃₋₃₃₉ epitope | peptide | self-assembling | linker toPEG | PEG | |
| | | linker | domain | | domain | |

*PEG is -(CH₂-CH₂-O)ₙ-CH₂-CH₂-OH

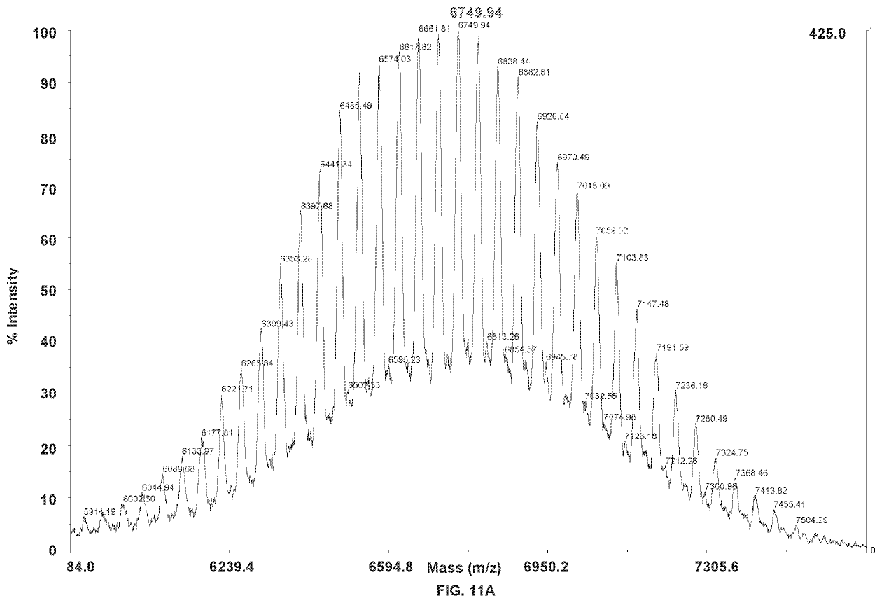

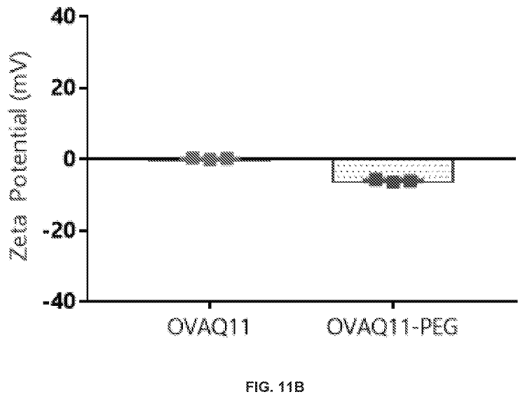

The inclusion of an additional immune peptide epitope conjugated to Q11 along with PEG could potentially disrupt or alter self-assembly. It was thought that adding PEG to a Q11-peptide epitope could potentially disrupt or alter the self-assembly of Q11 or the nanofiber. Furthermore, in a PEG-peptide design in which a cell adhesion motif was contained within the fibrillizing domain, the adhesion motif was suggested to be buried within the nanofiber core. We thus designed our material with the model peptide epitope OVA₃₂₃₋₃₃₉ connected via a flexible (Ser-Gly)₂ linker to the N-terminal of Q11, and a 3000 MW PEG block on the C-terminal, postulating that this would create a Q11 peptide core with OVA epitopes interspersed among the PEG corona. Due to the use of a polydisperse PEG block, the resulting PEG-peptide conjugate (OVAQ11-PEG) was polydisperse (FIG. 11A, FIG. 11B).

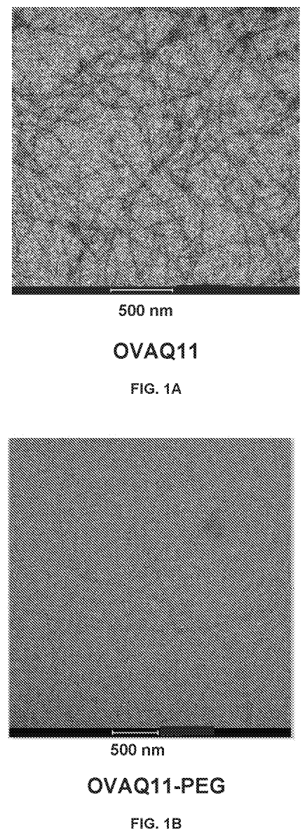

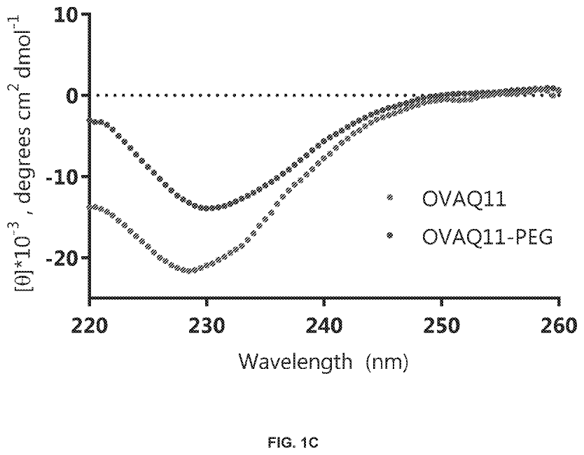

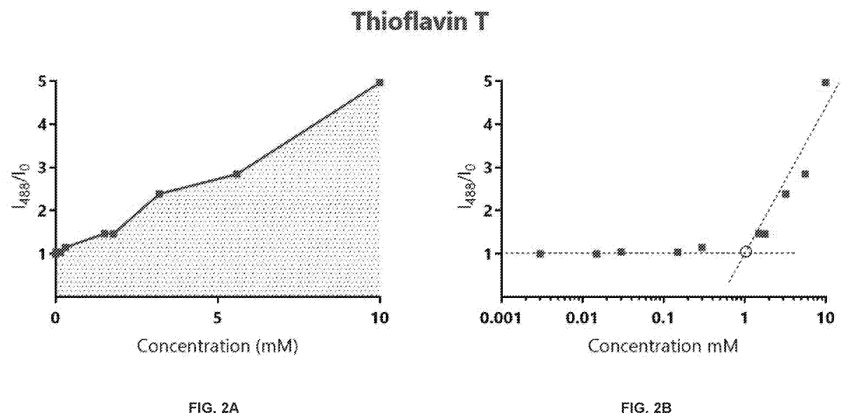

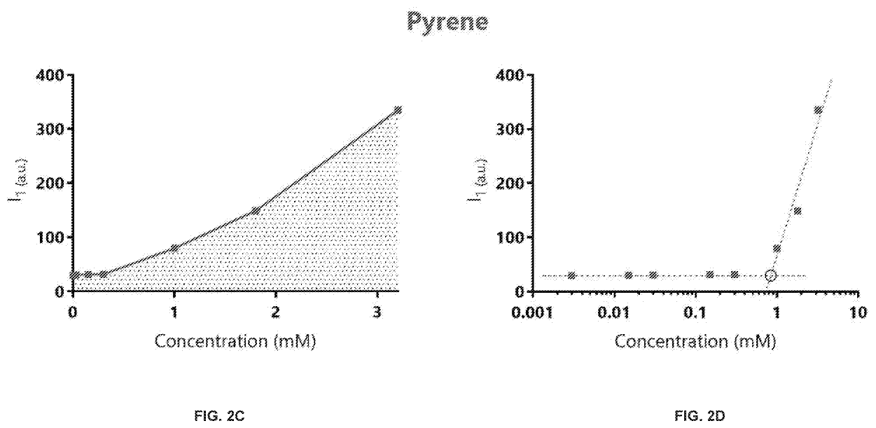

OVAQ11-PEG self-assembled in PBS to form high aspect ratio nanofibers morphologically similar to those observed for OVAQ11 (FIG. 1A, FIG. 1B, FIG. 1C). To characterize the secondary structure of these nanofibers, we used circular dichroism (CD) and fluorescent probes. The CD spectra of OVAQ11-PEG showed the same minima near 230 nm as previously reported for OVAQ11 (Rudra JS, et al. PNAS 2010, 107, 622-627), suggesting a similar β-sheet secondary structure. To confirm this finding, we employed two fluorescent probes, Thioflavin T (ThT) and pyrene, which have been used previously in the characterization of PEG-peptide conjugates. Thioflavin T binds to β-sheet rich structures, though our previous work with OEG-Q11 has shown that the presence of just three ethylene oxide units can diminish the overall magnitude of fluorescence. We thus measured the fluorescent signal of OVAQ11-PEG assembled at a range of concentrations in a solution of ThT in PBS. We observed a linear increase of fluorescence with peptide concentration above a critical aggregation concentration (c.a.c.) (FIG. 2A). To graphically estimate the c.a.c. of OVAQ11-PEG, we plotted the ThT fluorescence data against a log₁₀ concentration axis, using the method reported by Hamley and colleagues (Castelletto V, et al. European Polymer Journal 2013, 49, 2961-2967) (FIG. 2B).

We performed a similar procedure using pyrene, which is sensitive to changes in hydrophobicity. Upon assembly of OVAQ11-PEG, sequestration of hydrophobic amino acids, particularly phenylalanine, within the nanofiber core should decrease the hydrophobicity of the solution, leading to an increase in fluorescence of pyrene's first vibronic band. We observed similar results as for the ThT assay, and both estimated the c.a.c. of OVAQ11-PEG to be near 1 mM (FIG. 2C-FIG. 2D). All subsequent experiments were thus performed with peptide concentrations of at least 2 mM. Additionally, due to our previous report that large amounts of negative charge can diminish the immunogenicity of Q11 (Wen Y, et al. ACS Nano 2016 doi:10.1021/acsnano.6b03409), we measured the zeta potential of OVAQ11-PEG and found it to be close to neutral (FIG. 11A, FIG. 11B).

### EXAMPLE 3

### Reduction of Nanofiber Length and Aspect Ratio

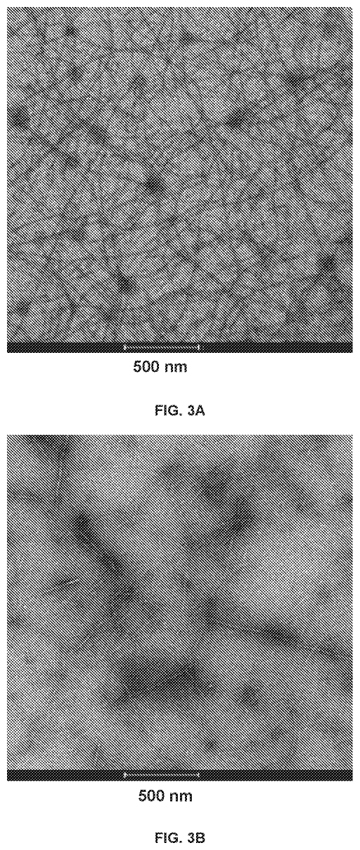

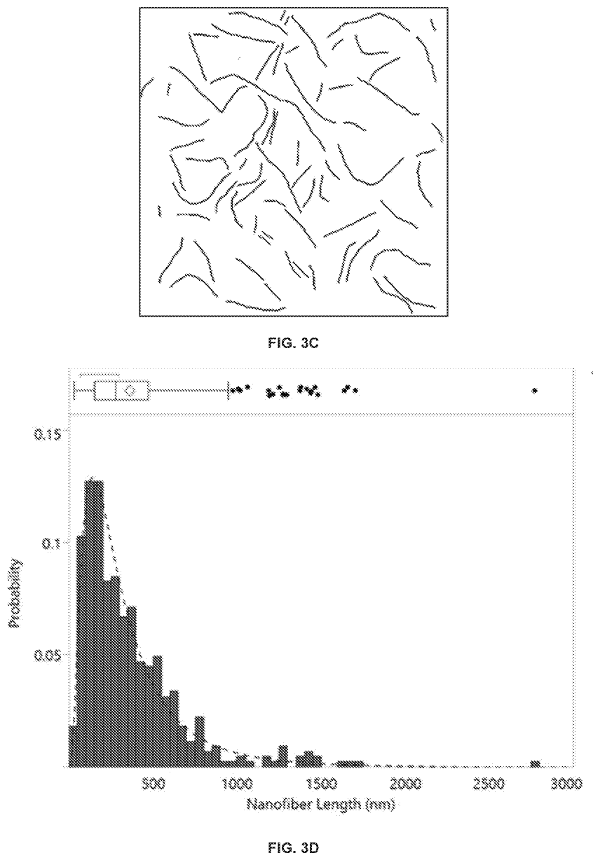

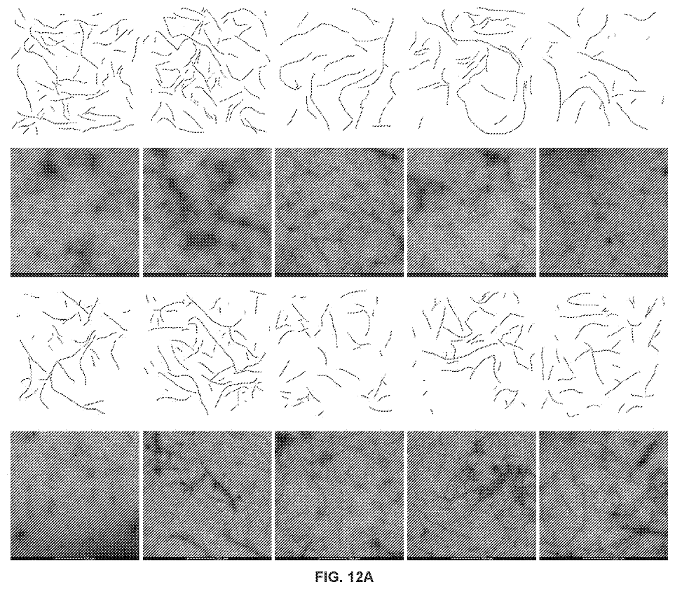

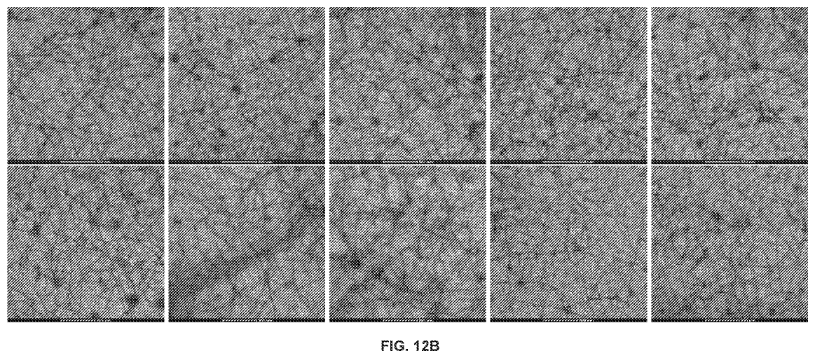

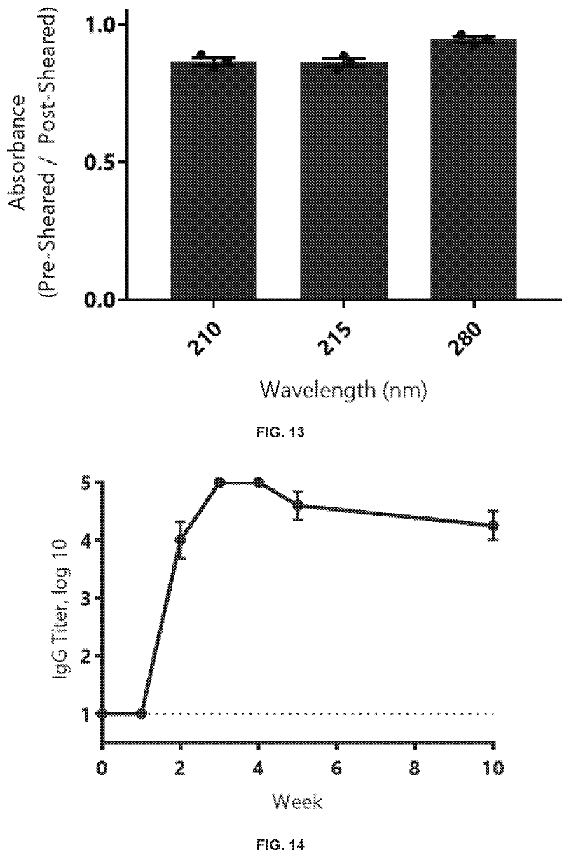

In addition to designing OVAQ11-PEG, we sought to test whether OVAQ11 could be used for sublingual immunization by reducing its size. While adhesive interactions between biomaterials and mucus are believed to be primarily responsible for inhibiting transport, the mucin network has defined pore sizes, suggesting that a material with a size below the pore size might transport more readily due to a reduction in steric hindrance. To test this hypothesis, we physically sheared assembled OVAQ11 nanofibers through a polycarbonate membrane containing 100 nm laser track-etched pores, a method previously reported to reduce the length of similar β-sheet peptides (Law B, et al. Bioconjugate Chemistry 2007, 18, 1701-1704; Wagh A, et al. Nanomedicine: Nanotechnology, Biology and Medicine 2013, 9, 449-457). To assess the effect of shearing on the length distribution of OVAQ11, we took 10 TEM images of OVAQ11 before and after shearing (FIG. 12A, FIG. 12B). The unsheared OVAQ11 nanofibers formed an entangled mat of nanofibers with lengths of microns, as previously reported by our group. In contrast, sheared OVAQ11 nanofibers varied in length but appeared significantly shorter. To quantify the length distribution, we used ImageJ to trace the length of 450 sheared nanofibers across the 10 images (FIG. 3A, FIG. 3B, FIG. 3C, FIG. 3D). The resulting distribution was roughly log-normal, with a median fiber length of 260 nm. Notably, the average pore size of human salivary mucus has been measured at 0.7 μm, and close to 90% of the sheared fibers were below this size. To test that shearing was not leading to a decrease in peptide concentration, we sheared an assembled tryptophan-labelled Q11 (W-Q11), and measured the absorbance at 210, 215, and 260 nm before and after shearing (FIG. 13).

We did not similarly quantify the distribution of sheared OVAQ11-PEG nanofibers. The competition between the assembly strength of the fibrillization domain and the tendency of PEG to crystallize upon drying samples prevents an accurate distribution from being measured via TEM, while the interaction between solvated PEG coronas in cryo-TEM makes it difficult to image a statistically significant number of fibers due to low density on the grids.

### EXAMPLE 4

### PEGylation and Shearing Does Not Diminish the Immunogenicity of OVAQ11

While our previous work has shown that OVAQ11 is highly immunogenic, we tested whether PEGylation or shearing compromises this immunogenicity. To study the question of the new materials' immunogenicity apart from the question of sublingual delivery, we tested sheared and PEGylated OVAQ11 in vitro with a cellular assay, and in vivo with subcutaneous immunization. As responses to Q11 have been shown to be T-cell dependent, it may be desired that the material can be taken up and the conjugated peptide epitope can be processed and presented on major histocompatibility (MHC) molecules to T cells. We incubated murine bone-marrow derived dendritic cells (BMDCs) with nanofibers in vitro for two hours to allow for uptake, then washed the cells and co-cultured the BMDCs with a reporter T-cell hybridoma whose T cell receptor is specific for the OVA epitope (DOBW cells), leading to the production of IL-2 (FIG. 4A). No significant difference in the concentration curves were observed between groups, suggesting that OVAQ11-PEG and sheared OVAQ11 could be efficiently internalized, processed, and presented.

We then immunized mice subcutaneously with OVAQ11-PEG or sheared OVAQ11-PEG, and compared the responses to that of OVAQ11. Mice were immunized with two 50 μL injections of 2 mM peptide, and boosted at week 4, leading to statistically indistinguishable titers of OVA-specific serum IgG (FIG. 4B). As Q11 has previously been shown to elicit Th2-biased phenotypes after subcutaneous immunization, we compared the antibody isotypes among groups (FIG. 4C). All groups showed a predominantly IgG1 response, suggestive of a Th2-biased response. Taken together, these experiments show that shearing and PEG conjugation do not diminish the immunogenicity of OVAQ11, and provide further evidence of the robustness of the Q11 platform to physical changes without loss of immunogenicity.

### EXAMPLE 5

### PEGylated, Sheared OVAQ11 Nanofibers Produce a Durable Antibody Response after Sublingual Immunization

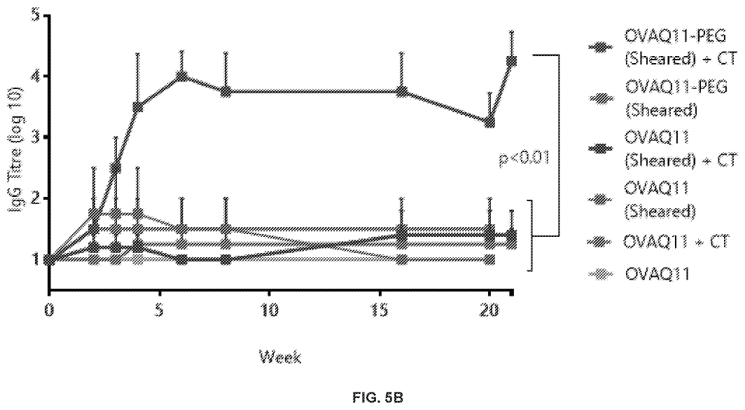

Having designed and characterized OVAQ11-PEG and observing no loss of immunogenicity, we next tested our hypothesis that these changes would enable sublingual immunization. Sublingual immunizations against split viruses or protein antigens have been achieved with only the inclusion of a mucosal adjuvant, such as cholera toxin (CT), in the formulation. However, sublingual immunization specifically against peptide epitopes is more challenging. Our experiences confirmed this, as we observed strong, durable responses against the full ovalbumin protein when formulated with CT and delivered sublingually (FIG. 14), but nearly no response to pOVA formulated with CT (FIG. 5A, FIG. 5B).

Strikingly, sublingual immunization of mice with sheared OVAQ11-PEG+CT led to high titers of OVA-specific serum IgG, while sheared or unsheared OVAQ11+CT failed to elicit stronger titers than pOVA+CT (FIG. 5B). Mice were immunized followed by boosts at 1 and 3 weeks after the primary immunization. The antibody response was extremely durable, as even four months after the second boost, mice maintained high levels of antigen-specific IgG in the serum. Furthermore, a recall response was observed after boosting mice at week 20, suggesting that immune memory may have developed. To minimize the potential confounding effects of mice swallowing material, we immunized deeply anesthetized mice with a conservative volume of only 7 μL. To show that OVAQ11-PEG, and not simply PEG conjugation, was necessary to promote the observed responses, we immunized mice sublingually with pOVA-PEG+CT as a control, and observed no response (FIG. 5A).

### EXAMPLE 6

### PEGylation, and not Shearing, is Critical for Eliciting Sublingual Immune Responses with OVAQ11

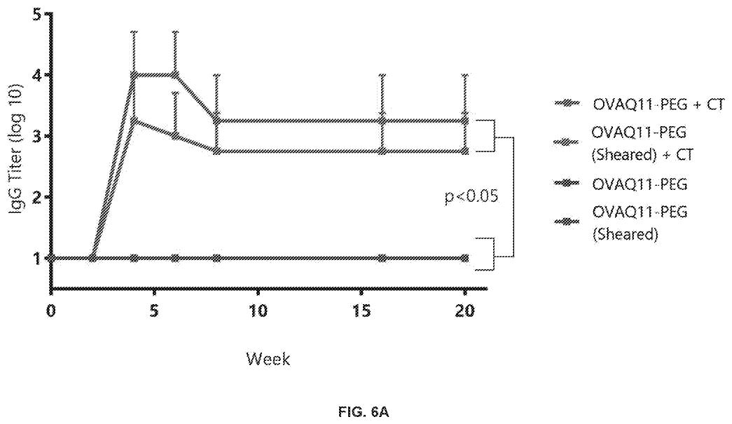

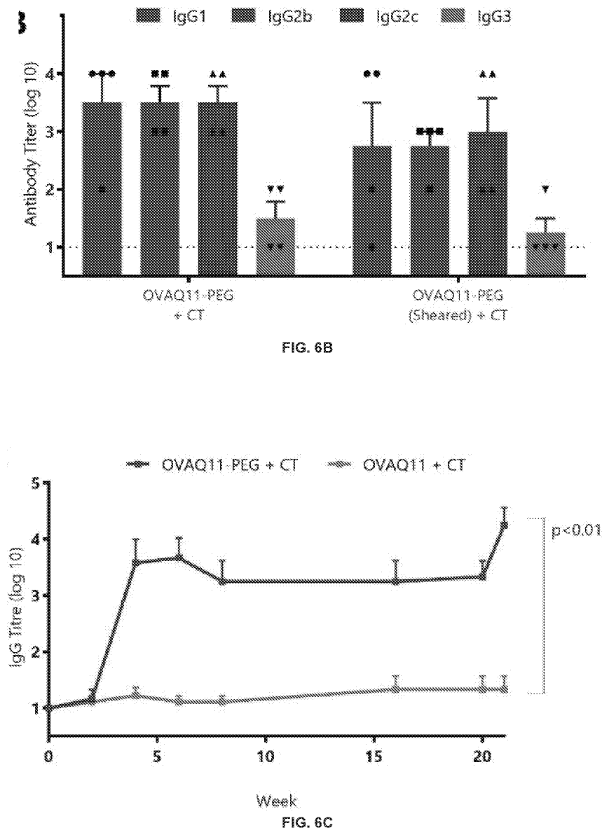

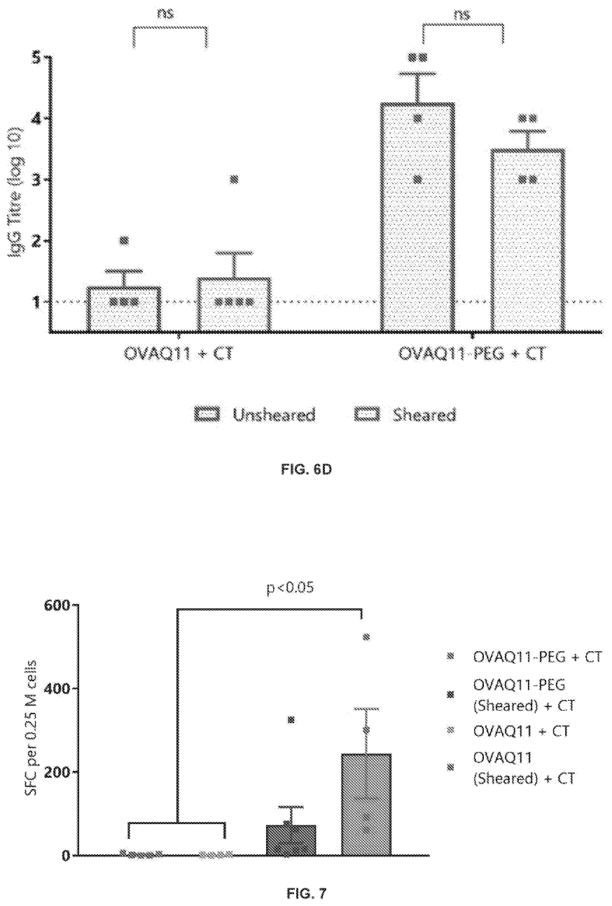

After discovering that the combined effects of shearing and PEGylation promoted strong sublingual immune responses, we sought to determine if one of these alterations was critical, or whether a synergistic effect exists. We immunized mice sublingually using sheared or unsheared OVAQ11-PEG, with or without cholera toxin (FIG. 6A, FIG. 6B, FIG. 6C, FIG. 6D). We found that sheared or unsheared OVAQ11-PEG led to strong OVA-specific antibody responses when formulated with cholera toxin. To highlight the effect of PEGylation on promoting sublingual immunization with OVAQ11, we combined data from sheared and unsheared groups and re-presented the data (FIG. 6C), showing the highly significant effect of PEGylation. Similarly, to show that shearing had no significant effect on the resulting antibody response, we compared the titers of sheared and unsheared groups and re-presented the data (FIG. 6D).

### EXAMPLE 7

### OVAQ11-PEG Elicited Antigen-Specific T-Cell Responses after Sublingual Immunization

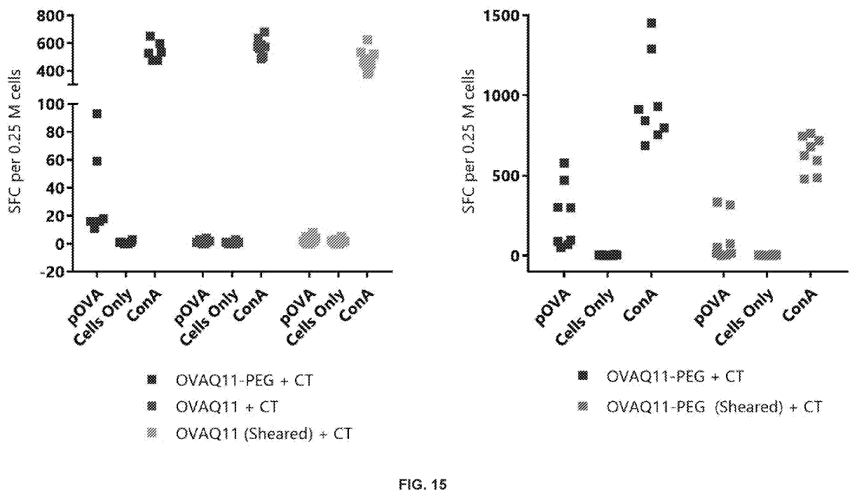

The OVA₃₂₃₋₃₃₉ epitope is unique in that it contains both B- and T-cell epitopes, which allowed us to simultaneously examine the ability of OVAQ11-PEG to elicit antigen-specific T-cell responses. We have previously shown that raising simultaneous T- and B-cell responses may be a factor both for optimizing responses to Q11 and for raising therapeutic immune responses (Mora-Solano C, et al. Biomaterials 2017, 149, 1-11; Pompano RR, et al. Adv. Healthc. Mater. 2014, 3, 1898-908). At week 21, one week after a final boost, mice were sacrificed and the spleens were harvested for analysis by ELISPOT (FIG. 7, FIG. 15). The results were consistent with the humoral responses described above, as mice immunized with OVAQ11-PEG+CT raised strong OVA-specific IFNy T cell responses and mice immunized with OVAQ11+CT raised no detectable responses. To our knowledge, this is the first report of raising antigen-specific T cell responses after sublingual peptide immunization.

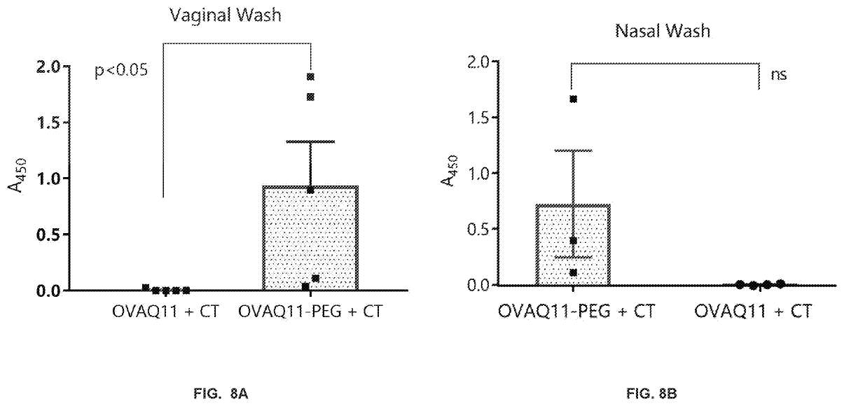

A benefit of mucosal vaccination routes is the ability to raise antibodies not only in the systemic circulation, but also at mucosal surfaces through which pathogens enter the body, where they can neutralize pathogens prior to entrance. The sublingual route has been shown to have a broader anatomical distribution of antibodies than other mucosal routes, with antibodies observed in both the upper and lower respiratory tracts and in the reproductive tract. Consistent with this, after sublingual immunization of mice with OVAQ11-PEG+CT we observed antigen-specific IgG in vaginal secretions, whereas no response was observed in mice immunized with OVAQ11+CT (FIG. 8A). We also observed responses in the nasal wash, suggestive of antibodies in the respiratory tract (FIG. 8B).

### EXAMPLE 8

### Sublingual Immunization with OVAQ11-PEG is Effective with Clinically Relevant Protein Adjuvant

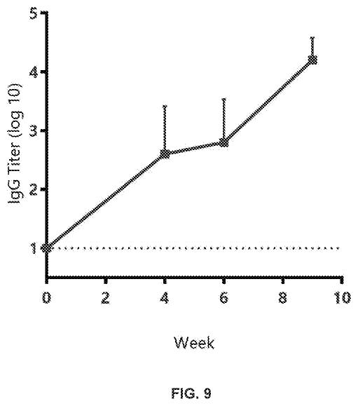

Our initial study of sublingual immunization with OVAQ11-PEG was performed using the model mucosal adjuvant CT, which is commonly employed in mouse models. After demonstrating that strong responses were raised with CT, we sought to use a more clinically translatable adjuvant. The B subunit of cholera toxin (CTB), which contains a strong T-cell epitope and interacts with the receptor GM1-ganglioside, has received significant attention as a mucosal adjuvant. CTB is approved for use in humans, as it is included in the oral cholera vaccine Dukoral and has been used as an intranasal adjuvant in human studies. Mice immunized with OVAQ11-PEG and the CTB protein raised antibody titers as strong as those with the full CT adjuvant after including an additional third boost at week 6 (FIG. 9).

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A peptide-polymer conjugate comprising: a self-assembling domain comprising a polypeptide and having a C-terminal and N-terminal end; a peptide epitope or protein antigen linked to the N-terminal end or the C-terminal end of the self-assembling domain; and a polyethylene glycol (PEG) domain linked to the other of the N-terminal end and the C-terminal end of the self-assembling domain.

Clause 2. The conjugate of clause 1, wherein the self-assembling domain comprises a polypeptide of 5 to 40 amino acids.

Clause 3. The conjugate of any one of the above clauses, wherein the self-assembling domain comprises a polypeptide having an amino acid sequence selected from SEQ ID NOs: 1-2 and 19-79.

Clause 4. The conjugate of any one of clauses 1-3, wherein the self-assembling domain forms at least one alpha helix.

Clause 5. The conjugate of any one of clauses 1-3, wherein the self-assembling domain forms at least one beta sheet.

Clause 6. The conjugate of any one of clauses 1-4, wherein the self-assembling domain comprises a polypeptide having an amino acid sequence of SEQ ID NO: 1 (QQKFQFQFEQQ).

Clause 7. The conjugate of any one of clauses 1-6, wherein the peptide-polymer conjugate comprises a peptide epitope, the peptide epitope comprising a polypeptide of 3 to 50 amino acids.

Clause 8. The conjugate of any one of clauses 1-6, wherein the peptide-polymer conjugate comprises a protein antigen, the protein antigen comprising a polypeptide of 10 to 500 amino acids.

Clause 9. The conjugate of any one of the above clauses, wherein the peptide epitope or protein antigen is linked to the N-terminal end of the self-assembling domain via a peptide linker.

Clause 10. The conjugate of clause 9, wherein the peptide linker comprises a polypeptide of 3 to 10 amino acids.

Clause 11. The conjugate of clause 9, wherein the peptide linker comprises a polypeptide having an amino acid sequence selected from SEQ ID NO: 4 (SGSG), SEQ ID NO: 9 (Gn wherein n is an integer from 1 to 10), SEQ ID NO: 10 (SGSGn wherein n is an integer from 1 to 10), SEQ ID NO: 11 (GSGS), SEQ ID NO: 12 (SSSS), SEQ ID NO: 13 (GGGS), SEQ ID NO: 14 (GGC), SEQ ID NO: 15 (GGS), SEQ ID NO: 16 ((GGC)8), SEQ ID NO: 17 (G4S)3, and SEQ ID NO: 18 (GGAAY).

Clause 12. The conjugate of any one of the above clauses, wherein the PEG domain has an average molecular weight of 300-5000 Da.

Clause 13. The conjugate of clause 12, wherein the PEG domain has an average molecular weight of 3000 Da.

Clause 14. The conjugate of any one of clauses 1-13, wherein the PEG domain is selected from —(CH2—CH2—O)n-CH2—CH2—OH, —(CH2—CH2—O)n—CH2—CH2—O—C1-4 alkyl, —(O—CH2—CH2)n—OH, and —(O—CH2—CH2)n—O—C1-4 alkyl, wherein n is an integer between 1 and 200.

Clause 15. The conjugate of clause 14, wherein the PEG domain is linked to the self-assembling domain via a linker.

Clause 16. The conjugate of any one of the above clauses, wherein the conjugate self-assembles into nanofibers.

Clause 17. The conjugate of any one of the above clauses, wherein the conjugate self-assembles into nanofibers having a length of 50 nm to 50,000 nm.

Clause 18. The conjugate of clause 16 or 17, wherein the nanofibers have uniform width.

Clause 19. The conjugate of clause 16 or 17, wherein the nanofibers have a width of 5-100 nm.

Clause 20. The conjugate of any one of the above clauses, wherein the conjugate elicits an antibody response upon or after administration to a subject.

Clause 21. The conjugate of any one of the above clauses, wherein the conjugate elicits a T cell response upon or after administration to a subject.

Clause 22. The conjugate of clause 20 or 21, wherein the conjugate is administered to the subject sublingually, orally, intranasally, intravenously, parenterally, subcutaneously, intramuscularly, intraperitoneally, rectally, intravaginally, or intrathecally.

Clause 23. The conjugate of clause 22, wherein the conjugate is administered to the subject sublingually.

Clause 24. A supramolecular complex comprising a plurality of the peptide-polymer conjugates of any one of clauses 1-23.

Clause 25. The supramolecular complex of clause 24, wherein the plurality of peptide-polymer conjugates comprises a plurality of identical peptide-polymer conjugates.

Clause 26. The supramolecular complex of clause 24, wherein the plurality of peptide-polymer conjugates comprises a plurality of non-identical peptide-polymer conjugates.

Clause 27. The supramolecular complex of clause 24, wherein the supramolecular complex comprises n different peptide-polymer conjugates, wherein n is an integer from 1 to 10,000.

Clause 28. The supramolecular complex of clause 27, wherein the n different peptide-polymer conjugates comprise different self-assembling domains from each other, different PEG domains from each other, or different peptide epitopes or protein antigens from each other, or a combination thereof.

Clause 29. The supramolecular complex of any one of clauses 25-28, further comprising at least one self-assembling domain not linked to a peptide epitope or a protein antigen, or at least one self-assembling domain not linked to a PEG domain, or at least one self-assembling domain not linked to a PEG domain and not linked to a peptide epitope or a protein antigen, or a combination thereof.

Clause 30. A composition comprising: a plurality of the conjugates of any one of clauses 1-23; and a pharmaceutically acceptable carrier.

Clause 31. A method of immunizing a subject, the method comprising administering to the subject an effective amount of the conjugate of any one of clauses 1-23, the supramolecular complex of any one of clauses 24-29, or the composition of clause 30.

Clause 32. A method of eliciting an immune response to a peptide epitope in a subject, the method comprising administering to the subject an effective amount of the conjugate of any one of clauses 1-23, the supramolecular complex of any one of clauses 24-29, or the composition of clause 30.

Clause 33. The method of clause 31 or 32, wherein the conjugate is administered to the subject sublingually, orally, intranasally, intravenously, parenterally, subcutaneously, intramuscularly, intraperitoneally, rectally, intravaginally, or intrathecally.

Clause 34. The method of clause 33, wherein the conjugate is administered sublingually.

Clause 35. The method of any one of clauses 33-34, wherein the conjugate is co-administered with an adjuvant.

Clause 36. The method of clause 35, wherein the adjuvant comprises cholera toxin, CpG, cyclic-di-GMP, cyclic-di-AMP, or a combination thereof.

SEQUENCES

SEQ ID NO: 1
Q11
QQKFQFQFEQQ
SEQ ID NO: 2
W-Q11
WSGSGQQKFQFQFEQQ
SEQ ID NO: 3
pOVA
ISQAVHAAHAEINEAGR
SEQ ID NO: 4
Linker
SGSG
SEQ ID NO: 5
OVAQ11
ISQAVHAAHAEINEAGRSGSGQQKFQFQFEQQ
SEQ ID NO: 6
OVAQ11-PEG
ISQAVHAAHAEINEAGRSGSGQQKFQFQFEQQ-X wherein X is
-NH-(CH₂-CH₂-O)ₙ-CH₂-
CH₂-OH and n is an integer between 1 and 200.
SEQ ID NO: 7
pOVA-PEG
ISQAVHAAHAEINEAGR-X wherein X is -NH-(CH₂-CH₂-O)ₙ-
CH₂-CH₂-OH and n is an integer between 1 and 200.
SEQ ID NO: 8
Biotin-pOVA
X-SGSG-ISQAVHAAHAEINEAGR wherein X is biotin.
SEQ ID NO: 9
Linker
Gₙ wherein n is an integer from 1 to 10)
SEQ ID NO: 10
Linker
SGSGₙ wherein n is an integer from 1 to 10)
SEQ ID NO: 11
Linker
GSGS
SEQ ID NO: 12
Linker
SSSS
SEQ ID NO: 13
Linker
GGGS
SEQ ID NO: 14
Linker
GGC
SEQ ID NO: 15
Linker
GGS
SEQ ID NO: 16
Linker
(GGC)₈
SEQ ID NO: 17
Linker
(G₄S)₃
SEQ ID NO: 18
Linker
GGAAY
SEQ ID NO: 19
ZₙbXXXbZₘ wherein b is independently any
positively charged amino acid, Z is
independently any amino acid, X is independently
any amino acid, n is an integer from 0 to 20,
and m is an integer from 0 to 20.
SEQ ID NO: 20
QARILEADAEILRAYARILEAHAEILRAQ
SEQ ID NO: 21
QAKILEADAEILKAYAKILEAHAEILKAQ
SEQ ID NO: 22
ADAEILRAYARILEAHAEILRAQ
SEQ ID NO: 23
EAK16-I
(AEAKAEAK)₂
SEQ ID NO: 24
EAK16-II
(AEAEAKAK)₂
SEQ ID NO: 25
EAK16-IV
(AEAE)₂(AKKE)₂
SEQ ID NO: 26
EMK16-II
(MEMEMKMK)
SEQ ID NO: 27
RAD16-I
(RADARADA)₂
SEQ ID NO: 28
RAD16-II
(RARARDRD)₂
SEQ ID NO: 29
RAD16-IV
(RARA)₂(RDRD)₂
SEQ ID NO: 30
DAR16-IV
(ADAD)₂(ARAR)₂
SEQ ID NO: 31
RAD8-I
(RADARADA)
SEQ ID NO: 32
RF6
(RFRFRF)
SEQ ID NO: 33
RF8
(RFRFRFRF)
SEQ ID NO: 34
KLD16
(KLDL)₄
SEQ ID NO: 35
FKFE2
(FKFE)₂
SEQ ID NO: 36
EFK12
(FKFE)₃
SEQ ID NO: 37
EFK16
(FEFEFKFK)₂
SEQ ID NO: 38
MAX1
VKVKVKVK-VDPPT-KVKVKVKV
SEQ ID NO: 39
MAX2 (V16T)
VKVKVKVK-VDPPT-KVKTKVKV
SEQ ID NO: 40
MAX3 (V7T, V16T)
VKVKVKTK-VDPPT-KVKTKVKV
SEQ ID NO: 41
MAX4
KVKVKVKV-KDPPS-VKVKVKVK
SEQ ID NO: 42
MAX5 (T12S)
VKVKVKVK-VDPPS-KVKVKVKV
SEQ ID NO: 43
MAX6 (V16E)
VKVKVKVK-VDPPT-KVKEKVKV
SEQ ID NO: 44
MAX7 (V16C)
VKVKVKVK-VDPPT-KVKCKVKV
SEQ ID NO: 45
MAX8 (K15E)
VKVKVKVK-VDPPT-KVEVKVKV
SEQ ID NO: 46
(K2E)
VEVKVKVK-VDPPT-KVKVKVKV
SEQ ID NO: 47
(K4E)
VKVEVKVK-VDPPT-KVKVKVKV
SEQ ID NO: 48
(K6E)
VKVKVEVK-VDPPT-KVKVKVKV
SEQ ID NO: 49
(K8E)
VKVKVKVE-VDPPT-KVKVKVKV
SEQ ID NO: 50
(K13E)
VKVKVKVK-VDPPT-EVKVKVKV
SEQ ID NO: 51
(K17E)
VKVKVKVK-VDPPT-KVKVEVKV
SEQ ID NO: 52
P11-1
QQRQQQQQEQQ
SEQ ID NO: 53
P11-2
QQRFQWQFEQQ
SEQ ID NO: 54
P11-3
QQRFQWQFQQQ
SEQ ID NO: 55
P11-4
QQRFEWEFEQQ
SEQ ID NO: 56
P11-5
QQOFOWOFQQQ
SEQ ID NO: 57
P11-7
SSRFSWSFESS
SEQ ID NO: 58
P11-8
QQRFOWOFEQQ
SEQ ID NO: 59
P11-9
SSRFEWEFESS
SEQ ID NO: 60
P11-12
SSRFOWOFESS
SEQ ID NO: 61
P11-14
QQOFOWOFOQQ
SEQ ID NO: 62
P11-16
NNRFOWOFEQQ
SEQ ID NO: 63
P11-18
TTRFOWOFETT
SEQ ID NO: 64
P11-19
QQRQOQOQEQQ
SEQ ID NO: 65
1
FEFEFKFKFEFEFKFK
SEQ ID NO: 66
2
FEFEAKFKFEFEFKFK
SEQ ID NO: 67
3
FEFEFKLKIEFEFKFK
SEQ ID NO: 68
4
FEAEVKLKIELEVKFK
SEQ ID NO: 69
5
GEAEVKLKIELEVKAK
SEQ ID NO: 70
6
GEAEVKIKIEVEAKGK
SEQ ID NO: 71
7
IEVEAKGKGEAEVKIK
SEQ ID NO: 72
8
IELEVKAKGEAEVKLK
SEQ ID NO: 73
9
IELEVKAKAEAEVKLK
SEQ ID NO: 74
10
IEAEGKGKIEGEAKIK
SEQ ID NO: 75
11
K₂(QL)₆K₂
SEQ ID NO: 76
12
E(QL)₆E
SEQ ID NO: 77
13
K₂(SL)₆K₂
SEQ ID NO: 78
14
E(SL)₆E
SEQ ID NO: 79
15
E(CLSL)₃E

1. A peptide-polymer conjugate comprising:
a self-assembling domain comprising a polypeptide and having a C-terminal and N-termina end;
a peptide epitope or protein antigen linked to the N-terminal end or the C-terminal end of the self-assembling domain; and
a polyethylene glycol (PEG) domain linked to the other of the N-terminal end and the C-terminal end of the self-assembling domain.

2. The conjugate of claim 1, wherein the self-assembling domain comprises a polypeptide of 5 to 40 amino acids.

3. The conjugate of claim 1, wherein the self-assembling domain comprises a polypeptide having an amino acid sequence selected from SEQ ID NOs: 1-2 and 19-79.

4. The conjugate of claim 1, wherein the self-assembling domain forms at least one alpha helix.

5. The conjugate of claim 1, wherein the self-assembling domain forms at least one beta sheet.

6. The conjugate of claim 1, wherein the self-assembling domain comprises a polypeptide having an amino acid sequence of SEQ ID NO: 1 (QQKFQFQFEQQ).

7. The conjugate of claim 1, wherein the peptide-polymer conjugate comprises a peptide epitope, the peptide epitope comprising a polypeptide of 3 to 50 amino acids.

8. The conjugate of claim 1, wherein the peptide-polymer conjugate comprises a protein antigen, the protein antigen comprising a polypeptide of 10 to 500 amino acids.

9. The conjugate of claim 1, wherein the peptide epitope or protein antigen is linked to the N-terminal end of the self-assembling domain via a peptide linker.

10. (canceled)

11. The conjugate of claim 9, wherein the peptide linker comprises a polypeptide having an amino acid sequence selected from SEQ ID NO: 4 (SGSG), SEQ ID NO: 9 (Gₙ wherein n is an integer from 1 to 10), SEQ ID NO: 10 (SGSGₙ wherein n is an integer from 1 to 10), SEQ ID NO: 11 (GSGS), SEQ ID NO: 12 (SSSS), SEQ ID NO: 13 (GGGS), SEQ ID NO: 14 (GGC), SEQ ID NO: 15 (GGS), SEQ ID NO: 16 ((GGC)₈), SEQ ID NO: 17 (G₄5)₃, and SEQ ID NO: 18 (GGAAY).

12. The conjugate of claim 1, wherein the PEG domain has an average molecular weight of 300-5000 Da.

13. (canceled)

14. The conjugate of claim 1, wherein the PEG domain is selected from —(CH₂—CH₂—O)ₙ—CH₂—CH₂—OH, —(CH₂—CH₂—O)ₙ—CH₂—CH₂—O—C₁₋₄ alkyl, —(O—CH₂—CH₂)ₙ—OH, and —(O—CH₂—CH₂)ₙ—O—C₁₋₄ alkyl, wherein n is an integer between 1 and 200.

15. (canceled)

16. The conjugate of claim 1, wherein the conjugate self-assembles into nanofibers.

17. The conjugate of claim 1, wherein the conjugate self-assembles into nanofibers having a length of 50 nm to 50,000 nm.

18. The conjugate of claim 16 or 17, wherein the nanofibers have uniform width of 5-100 nm.

19. (canceled)

20. The conjugate of claim 1, wherein the conjugate elicits an antibody response upon or after administration to a subject.

21. The conjugate of claim 1, wherein the conjugate elicits a T cell response upon or after administration to a subject. 22-23. (canceled)

24. A supramolecular complex comprising a plurality of the peptide-polymer conjugates of claim 1.

25. The supramolecular complex of claim 24, wherein the plurality of peptide-polymer conjugates comprises a plurality of identical peptide-polymer conjugates.

26. The supramolecular complex of claim 24, wherein the plurality of peptide-polymer conjugates comprises a plurality of non-identical peptide-polymer conjugates.

27. The supramolecular complex of claim 24, wherein the supramolecular complex comprises n different peptide-polymer conjugates, wherein n is an integer from 1 to 10,000.

28. The supramolecular complex of claim 27, wherein the n different peptide-polymer conjugates comprise different self-assembling domains from each other, different PEG domains from each other, or different peptide epitopes or protein antigens from each other, or a combination thereof.

29. The supramolecular complex of claim 25, further comprising at least one self-assembling domain not linked to a peptide epitope or a protein antigen, or at least one self-assembling domain not linked to a PEG domain, or at least one self-assembling domain not linked to a PEG domain and not linked to a peptide epitope or a protein antigen, or a combination thereof.

30. A composition comprising:
a plurality of the conjugates of claim 1; and
a pharmaceutically acceptable carrier.

31. A method of immunizing a subject, the method comprising administering to the subject an effective amount of the conjugate of claim 1.

32. A method of eliciting an immune response to a peptide epitope in a subject, the method comprising administering to the subject an effective amount of the conjugate of claim 1.

33-34. (canceled)

35. The method of claim 31, wherein the conjugate is co-administered with an adjuvant.

36. The method of claim 35, wherein the adjuvant comprises cholera toxin, CpG, cyclic-di-GMP, cyclic-di-AMP, or a combination thereof.


SEQUENCE LISTING (79 sequences)

SEQ ID NO: 1 - protein, 11 aa, Artificial Sequence
  Synthetic
QQKFQFQFEQQ

SEQ ID NO: 2 - protein, 16 aa, Artificial Sequence
  Synthetic
WSGSGQQKFQFQFEQQ

SEQ ID NO: 3 - protein, 17 aa, Artificial Sequence
  Synthetic
ISQAVHAAHAEINEAGR

SEQ ID NO: 4 - protein, 4 aa, Artificial Sequence
  Synthetic
SGSG

SEQ ID NO: 5 - protein, 32 aa, Artificial Sequence
  Synthetic
ISQAVHAAHAEINEAGRSGSGQQKFQFQFEQQ

SEQ ID NO: 6 - protein, 33 aa, Artificial Sequence
  Synthetic
  Xaa (33)..(33): Xaa is -NH-(CH2-CH2-O)n-CH2-CH2-OH and n is an integer between 1 and 200
ISQAVHAAHAEINEAGRSGSGQQKFQFQFEQQX

SEQ ID NO: 7 - protein, 18 aa, Artificial Sequence
  Synthetic
  Xaa (18)..(18): Xaa is -NH-(CH2-CH2-O)n-CH2-CH2-OH and n is an integer between 1 and 200
ISQAVHAAHAEINEAGRX

SEQ ID NO: 8 - protein, 21 aa, Artificial Sequence
  Synthetic
SGSGISQAVHAAHAEINEAGR

SEQ ID NO: 9 - protein, 1 aa, Artificial Sequence
  Synthetic
  Gly (1)..(1): Gly may repeat from 1 to 10 times
G

SEQ ID NO: 10 - protein, 4 aa, Artificial Sequence
  Synthetic
  Ser Gly Ser Gly (1)..(4): Ser Gly Ser Gly may repeat from 1 to 10 times
SGSG

SEQ ID NO: 11 - protein, 4 aa, Artificial Sequence
  Synthetic
GSGS

SEQ ID NO: 12 - protein, 4 aa, Artificial Sequence
  Synthetic
SSSS

SEQ ID NO: 13 - protein, 4 aa, Artificial Sequence
  Synthetic
GGGS

SEQ ID NO: 14 - protein, 3 aa, Artificial Sequence
  Synthetic
GGC

SEQ ID NO: 15 - protein, 3 aa, Artificial Sequence
  Synthetic
GGS

SEQ ID NO: 16 - protein, 24 aa, Artificial Sequence
  Synthetic
GGCGGCGGCGGCGGCGGCGGCGGC

SEQ ID NO: 17 - protein, 15 aa, Artificial Sequence
  Synthetic
GGGGSGGGGSGGGGS

SEQ ID NO: 18 - protein, 5 aa, Artificial Sequence
  Synthetic
GGAAY

SEQ ID NO: 19 - protein, 7 aa, Artificial Sequence
  Synthetic
  Xaa (1)..(1): Xaa at position 1 is any amino acid, repeated 0 to 20 times
  Xaa (2)..(2): Xaa at position 2 is any positively charged amino acid
  Xaa (3)..(5): Xaa at positions 3-5 are independently any amino acid
  Xaa (6)..(6): Xaa at position 6 is any positively charged amino acid
  Xaa (7)..(7): Xaa at position 7 is any amino acid, repeated 0 to 20 times
XXXXXXX

SEQ ID NO: 20 - protein, 29 aa, Artificial Sequence
  Synthetic
QARILEADAEILRAYARILEAHAEILRAQ

SEQ ID NO: 21 - protein, 29 aa, Artificial Sequence
  Synthetic
QAKILEADAEILKAYAKILEAHAEILKAQ

SEQ ID NO: 22 - protein, 23 aa, Artificial Sequence
  Synthetic
ADAEILRAYARILEAHAEILRAQ

SEQ ID NO: 23 - protein, 16 aa, Artificial Sequence
  Synthetic
AEAKAEAKAEAKAEAK

SEQ ID NO: 24 - protein, 16 aa, Artificial Sequence
  Synthetic
AEAEAKAKAEAEAKAK

SEQ ID NO: 25 - protein, 16 aa, Artificial Sequence
  Synthetic
AEAEAEAEAKKEAKKE

SEQ ID NO: 26 - protein, 8 aa, Artificial Sequence
  Synthetic
MEMEMKMK

SEQ ID NO: 27 - protein, 16 aa, Artificial Sequence
  Synthetic
RADARADARADARADA

SEQ ID NO: 28 - protein, 16 aa, Artificial Sequence
  Synthetic
RARARDRDRARARDRD

SEQ ID NO: 29 - protein, 16 aa, Artificial Sequence
  Synthetic
RARARARARDRDRDRD

SEQ ID NO: 30 - protein, 16 aa, Artificial Sequence
  Synthetic
ADADADADARARARAR

SEQ ID NO: 31 - protein, 8 aa, Artificial Sequence
  Synthetic
RADARADA

SEQ ID NO: 32 - protein, 6 aa, Artificial Sequence
  Synthetic
RFRFRF

SEQ ID NO: 33 - protein, 8 aa, Artificial Sequence
  Synthetic
RFRFRFRF

SEQ ID NO: 34 - protein, 16 aa, Artificial Sequence
  Synthetic
KLDLKLDLKLDLKLDL

SEQ ID NO: 35 - protein, 8 aa, Artificial Sequence
  Synthetic
FKFEFKFE

SEQ ID NO: 36 - protein, 12 aa, Artificial Sequence
  Synthetic
FKFEFKFEFKFE

SEQ ID NO: 37 - protein, 16 aa, Artificial Sequence
  Synthetic
FEFEFKFKFEFEFKFK

SEQ ID NO: 38 - protein, 21 aa, Artificial Sequence
  Synethic
VKVKVKVKVDPPTKVKVKVKV

SEQ ID NO: 39 - protein, 21 aa, Artificial Sequence
  Synthetic
VKVKVKVKVDPPTKVKTKVKV

SEQ ID NO: 40 - protein, 21 aa, Artificial Sequence
  Synthetic
VKVKVKTKVDPPTKVKTKVKV

SEQ ID NO: 41 - protein, 21 aa, Artificial Sequence
  Synthetic
KVKVKVKVKDPPSVKVKVKVK

SEQ ID NO: 42 - protein, 21 aa, Artificial Sequence
  Synthetic
VKVKVKVKVDPPSKVKVKVKV

SEQ ID NO: 43 - protein, 21 aa, Artificial Sequence
  Synthetic
VKVKVKVKVDPPTKVKEKVKV

SEQ ID NO: 44 - protein, 21 aa, Artificial Sequence
  Synthetic
VKVKVKVKVDPPTKVKCKVKV

SEQ ID NO: 45 - protein, 21 aa, Artificial Sequence
  Synthetic
VKVKVKVKVDPPTKVEVKVKV

SEQ ID NO: 46 - protein, 21 aa, Artificial Sequence
  Synthetic
VEVKVKVKVDPPTKVKVKVKV

SEQ ID NO: 47 - protein, 21 aa, Artificial Sequence
  Synthetic
VKVEVKVKVDPPTKVKVKVKV

SEQ ID NO: 48 - protein, 21 aa, Artificial Sequence
  Synthetic
VKVKVEVKVDPPTKVKVKVKV

SEQ ID NO: 49 - protein, 21 aa, Artificial Sequence
  Synthetic
VKVKVKVEVDPPTKVKVKVKV

SEQ ID NO: 50 - protein, 21 aa, Artificial Sequence
  Synthetic
VKVKVKVKVDPPTEVKVKVKV

SEQ ID NO: 51 - protein, 21 aa, Artificial Sequence
  Synthetic
VKVKVKVKVDPPTKVKVEVKV

SEQ ID NO: 52 - protein, 11 aa, Artificial Sequence
  Synthetic
QQRQQQQQEQQ

SEQ ID NO: 53 - protein, 11 aa, Artificial Sequence
  Synthetic
QQRFQWQFEQQ

SEQ ID NO: 54 - protein, 11 aa, Artificial Sequence
  Synthetic
QQRFQWQFQQQ

SEQ ID NO: 55 - protein, 11 aa, Artificial Sequence
  Synthetic
QQRFEWEFEQQ

SEQ ID NO: 56 - protein, 11 aa, Artificial Sequence
  Synthetic
  Xaa (3)..(3): Xaa at position 3 is Ornithine
  Xaa (5)..(5): Xaa at position 5 is Ornithine
  Xaa (7)..(7): Xaa at position 7 is Ornithine
QQXFXWXFQQQ

SEQ ID NO: 57 - protein, 11 aa, Artificial Sequence
  Synthetic
SSRFSWSFESS

SEQ ID NO: 58 - protein, 11 aa, Artificial Sequence
  Synthetic
  Xaa (5)..(5): Xaa at position 5 is Ornithine
  Xaa (7)..(7): Xaa at position 7 is Ornithine
QQRFXWXFEQQ

SEQ ID NO: 59 - protein, 11 aa, Artificial Sequence
  Synthetic
SSRFEWEFESS

SEQ ID NO: 60 - protein, 11 aa, Artificial Sequence
  Synthetic
  Xaa (5)..(5): Xaa at position 5 is Ornithine
  Xaa (7)..(7): Xaa at position 7 is Ornithine
SSRFXWXFESS

SEQ ID NO: 61 - protein, 11 aa, Artificial Sequence
  Synthetic
  Xaa (3)..(3): Xaa at position 3 is Ornithine
  Xaa (5)..(5): Xaa at position 5 is Ornithine
  Xaa (7)..(7): Xaa at position 7 is Ornithine
  Xaa (9)..(9): Xaa at position 9 is Ornithine
QQXFXWXFXQQ

SEQ ID NO: 62 - protein, 11 aa, Artificial Sequence
  Synthetic
  Xaa (5)..(5): Xaa at position 5 is Ornithine
  Xaa (7)..(7): Xaa at position 7 is Ornithine
NNRFXWXFEQQ

SEQ ID NO: 63 - protein, 11 aa, Artificial Sequence
  Synthetic
  Xaa (5)..(5): Xaa at position 5 is Ornithine
  Xaa (7)..(7): Xaa at position 7 is Ornithine
TTRFXWXFETT

SEQ ID NO: 64 - protein, 11 aa, Artificial Sequence
  Synthetic
  Xaa (5)..(5): Xaa at position 5 is Ornithine
  Xaa (7)..(7): Xaa at position 7 is Ornithine
QQRQXQXQEQQ

SEQ ID NO: 65 - protein, 16 aa, Artificial Sequence
  Synthetic
FEFEFKFKFEFEFKFK

SEQ ID NO: 66 - protein, 16 aa, Artificial Sequence
  Synthetic
FEFEAKFKFEFEFKFK

SEQ ID NO: 67 - protein, 16 aa, Artificial Sequence
  Synthetic
FEFEFKLKIEFEFKFK

SEQ ID NO: 68 - protein, 16 aa, Artificial Sequence
  Synthetic
FEAEVKLKIELEVKFK

SEQ ID NO: 69 - protein, 16 aa, Artificial Sequence
  Synthetic
GEAEVKLKIELEVKAK

SEQ ID NO: 70 - protein, 16 aa, Artificial Sequence
  Synthetic
GEAEVKIKIEVEAKGK

SEQ ID NO: 71 - protein, 16 aa, Artificial Sequence
  Synthetic
IEVEAKGKGEAEVKIK

SEQ ID NO: 72 - protein, 16 aa, Artificial Sequence
  Synthetic
IELEVKAKGEAEVKLK

SEQ ID NO: 73 - protein, 16 aa, Artificial Sequence
  Synthetic
IELEVKAKAEAEVKLK

SEQ ID NO: 74 - protein, 16 aa, Artificial Sequence
  Synthetic
IEAEGKGKIEGEAKIK

SEQ ID NO: 75 - protein, 16 aa, Artificial Sequence
  Synthetic
KKQLQLQLQLQLQLKK

SEQ ID NO: 76 - protein, 14 aa, Artificial Sequence
  Synthetic
EQLQLQLQLQLQLE

SEQ ID NO: 77 - protein, 16 aa, Artificial Sequence
  Synthetic
KKSLSLSLSLSLSLKK

SEQ ID NO: 78 - protein, 14 aa, Artificial Sequence
  Synthetic
ESLSLSLSLSLSLE

SEQ ID NO: 79 - protein, 14 aa, Artificial Sequence
  Synthetic
ECLSLCLSLCLSLE